(19) United States
(12) Patent Application Publication
(10) Pub. No.: US 2010/0256086 A1
(43) Pub. Date: Oct. 7, 2010

(54) NON-HYDROLYZABLE NUCLEOSIDE DI- OR TRI-PHOSPHATE DERIVATIVES AND USES THEREOF

(75) Inventor: Bilha FISCHER, Shoham (IL)

Correspondence Address: BROWDY AND NEIMARK, P.L.L.C.;624 NINTH STREET, NW, SUITE 300, WASHINGTON, DC, 20001-5303, US

(73) Assignee: BAR-ILAN UNIVETRSITY, Ramat Gan (IL)

(21) Appl. No.: 12/744,473

(22) PCT Filed: Nov. 23, 2008
(86) PCT No.: PCT/IL08/01535

§ 371 Date: May 24, 2010

Related U.S. Application Data
(60) Provisional application No. 60/989,888, filed on Nov. 23, 2007.

(51) Int. Cl. A61K 31/7076; C07H 19/20; A61P 3/10

(52) U.S. Cl. 514/47; 536/26.21

(57) ABSTRACT

The invention provides non-hydrolyzable nucleoside polyphosphate derivatives, e.g., 2MeS-adenosine-β,γ-CH₂-5′-O-(1-boranotriphosphate), 2MeS-adenosine-β,γ-CCl₂-5′-O-(1-boranotriphosphate), 2-MeS-adenosine-5′-dichloro methylene-diphosphate, 2-MeS-adenosine-5′-difluoromethylene-diphosphate and 2MeS-adenosine-5′-O-(1-boranodiphosphate), as well as pharmaceutical compositions thereof. These compounds are useful for prevention or treatment of diseases or disorders modulated by P2Y-receptors such as type 2 diabetes, and for pain control.

### FIELD OF THE INVENTION

The present invention relates to non-hydrolyzable nucleoside polyphosphate derivatives and to pharmaceutical compositions comprising them. The compounds are useful for prevention or treatment of diseases or disorders modulated by P2Y-receptors such as type 2 diabetes, and for pain control.

### BACKGROUND OF THE INVENTION

The P2 receptor (P2R) superfamily, consisting of ligand-gated ion-channels (P2XRs) and G protein-coupled receptors (P2YRs), are activated principally by the extracellular nucleotides ATP, ADP, UTP or UDP (Jacobson et al., 2002). In addition, P2 receptors are activated by several dinucleoside polyphosphates (dinucleotides) (WO 2003/0207825; Shaver et al., 2005).

P2YRs are attractive pharmaceutical targets due to their involvement in the modulation of various functions in many tissues and organs under both normal and pathophysiological conditions (Williams and Jarvis, 2000; Guile et al., 2001; Fischer, 1999), thus making P2YR agonists potential drugs. Currently, P2YR agonists proposed as drugs consist of a nucleotide scaffold (Williams and Jarvis, 2000; Fischer, 1999; Abbracchio et al., 2006; Jacobson et al., 2002; Laxman and Beavo, 2007) that is enzymatically and chemically unstable.

Approaches to overcome the inherent instability of nucleotide-based drug candidates include the use of (i) dinucleotides that are metabolically more stable than the corresponding nucleotides; (ii) non-nucleotide P2R ligands; (iii) nucleotide pro-drugs; and (iv) isoster-based non-hydrolyzable nucleotides.

The first approach is rather promising and indeed several dinucleotides have been administered in human pre-clinical trials. For instance, Ap₄A, Up₄U and Up₄dC have been proven effective for lowering blood pressure during anesthesia, and as a treatment for dry eye disease, cystic fibrosis and retinal detachment, respectively (Kikuta et al., 1999; Maminishkis et al., 2002; Mundasad et al., 2001; Yerxa et al., 2002).

The second approach has been successful in the case of Clopidogrel (Plavix®, Sanofi-Synthelabo/BMS), a platelet anti-aggregating agent used for the prevention of secondary vascular events (Chow and Ziegelstein, 2007), which is the only P2YR targeting drug currently available. Clopidogrel, acting as a P2Y₁₂ receptor antagonist (Angiolillo et al., 2006a and 2006b), is a non-nucleotide.

The third approach involves the preparation of masked triester nucleotide prodrugs. These prodrugs, e.g., the anti-HIV nucleoside analogue d4T, proved membrane soluble and released the active nucleotide within the cell (McGuigan et al., 1993, 1996a and 1996b; WO/2002/055521).

Only a few attempts to improve the stability of nucleotide-based drug candidates, either enzyme inhibitors or receptor ligands, by the bioisoster approach have been reported (Blackburn et al., 1987; Cusack et al., 1987; He et al., 1997; Kowalska et al., 2007; Lin et al., 2001; Misiura et al., 2005; Romaniuk and Eckstein, 1981; Stingelin et al., 1980).

Diabetes mellitus is one of the most prevalent chronic diseases in the Western world, affecting up to 5% of the population. It is a heterogeneous group of disorders characterized by a chronic hyperglycemia—resulting from defects in insulin secretion, insulin action, or a combination of both—with additional abnormalities in lipid and protein metabolism. In addition to its chronic metabolic abnormalities, diabetes is associated with long-term complications involving various organs, especially the eyes, nerves, blood vessels, heart and kidney, which may result in blindness, amputations, cardiovascular disease and end stage renal disease. The development of diabetic complications appears to be related to the chronic elevation of blood glucose. There is no current cure for diabetes; however, effective glycemic control can lower the incidence of diabetic complications and reduce their severity.

Type 2 diabetes, also termed non-insulin-dependent diabetes mellitus (NIDDM), affects approximately 95% of patients with diabetes and appears to be a complex polygenic disease in which insulin resistance and relative insulin deficiency coexist. Thus, improvement of insulin secretion is a major therapeutic goal. The deficiency of insulin release expresses itself not only by the absence of first-phase insulin response to glucose, but also by a global reduction in the magnitude of insulin release to 10-20% of the normal secretory capacity. Patients with type 2 diabetes are treated with various oral antidiabetic agents, insulin injections or a combination of both. The currently available oral antidiabetic drugs are targeted either to increasing insulin secretion from the pancreatic beta-cells, reducing peripheral insulin resistance, or to slowing the absorption of carbohydrates from the intestine.

Approximately half of the patients with type 2 diabetes are treated with oral agents, a considerable proportion of them with agents that stimulate insulin secretion. The choice of insulin secretagogues is limited to the sulfonylureas and related compounds (“glinides”), which elicit insulin secretion by binding to a regulatory subunit of membrane ATP-sensitive potassium channel, inducing its closure. However, sulfonylureas have several undesired effects in addition to possible long-term adverse effect on their specific target, the pancreatic beta-cells. These side-effects include the risk of hypoglycemia due to stimulation of insulin secretion at low glucose concentrations, the difficulty of achieving normal glycemia in a significant number of patients, the annually 5-10% secondary failure rate of adequate glycemic control, and possible negative effects on the cardiovascular system.

The presence of P2YRs on pancreatic beta cells is well documented and their activation results in stimulation of insulin secretion at stimulating glucose concentrations. The mechanism whereby P2YR agonists enhance glucose-induced insulin release may involve the cyclic AMP/Protein Kinase A signaling pathway, which has been reported to increase the effectiveness of the K⁺_{ATP} channel-independent action of glucose.

Various P2R selective ligands have been shown to increase insulin secretion and decrease glycemia in vivo. The list of ligands includes 2-methylthio-ATP, which breaks down rapidly into 2-MeS-adenosine and thus was injected directly to the pancreatico-duodenal artery, and adenosine 5′-O-(2-thio)diphosphate, which is stable to enzymatic hydrolysis and thus was administered either intravenously or orally.

Almost all current synthetic P2-receptor agonists are modifications of the ATP or UTP pharmacophore. The purine (pyrimidine) ring system, the ribose moiety, or the triphosphate chain are modified at one or more positions (Fischer, 1999). Previously, we have reported the synthesis of ATP derivatives bearing a long thioether substitution at C-2 position, such as 2-thioether-5′-O-(1-thiotriphosphate) adenosine derivatives (Fischer et al., 1999).

WO 2003/034978, corresponding to U.S. Pat. No. 7,319,093, discloses a series of potent and selective P2Y₁R agonists based on boranophosphate isosters of ATP analogues (adenosine-5′-α-borano-triphosphate analogues) (Nahum et al., 2002; Major et al., 2004; Tulapurkar et al., 2004; Farret et al., 2006). These analogues proved to be highly stable at physiological pH and relatively stable at pH 1.4 and 37° C. Furthermore, these agonists were relatively resistant to hydrolysis by ecto-nucleoside triphosphate diphosphohydrolase (e-NTPDase) and proved to be highly potent insulin secretagogues at perfused rat pancreas. The most effective agonist was 2-MeS-ATP-α-B, 1, which induced a 9-fold enhancement of insulin secretion as compared to basal secretion with an EC₅₀ of 28 nM. The insulin-releasing action of 2-MeS-ATP-α-B is glucose-dependent, suggesting that this compound could be a drug candidate for treatment of type-2 diabetes; however, the observation that it is unstable to alkaline phosphatase disqualified this compound for use as a drug.

### SUMMARY OF THE INVENTION

In one aspect, the present invention relates to a compound of the general formula I:

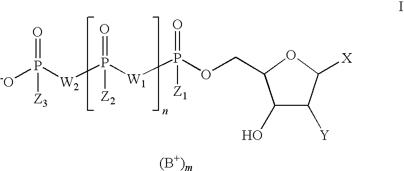

wherein

X is an adenine residue of the formula Ia, linked through the 9-position:

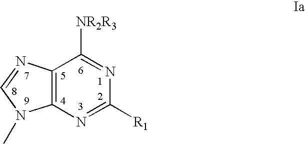

wherein

R₁ is H, halogen, O-hydrocarbyl, S-hydrocarbyl, NR₄R₅, heteroaryl, unsubstituted hydrocarbyl or hydrocarbyl substituted by halogen, CN, SCN, NO₂, OR₄, SR₄, NR₄R₅ or heteroaryl, wherein R₄ and R₅ each independently is H or hydrocarbyl or R₄ and R₅ together with the nitrogen atom to which they are attached form a 5- or 6-membered saturated or unsaturated heterocyclic ring optionally containing 1-2 further heteroatoms selected from oxygen, nitrogen or sulfur, the additional nitrogen being unsubstituted or substituted by alkyl substituted by halogen, hydroxyl or phenyl; and

R₂ and R₃ each independently is H or hydrocarbyl;

or X is an uracil residue of the formula Ib, linked through the 1-position:

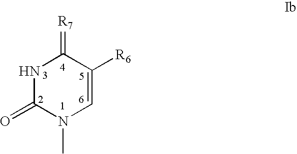

wherein

R₆ is H, halogen, O-hydrocarbyl, S-hydrocarbyl, NR₈R₉, heteroaryl, unsubstituted hydrocarbyl or hydrocarbyl substituted by halogen, CN, SCN, NO₂, OR₈, SR₈, NR₈R₉ or heteroaryl, wherein R₈ and R₉ each independently is H or hydrocarbyl or R₈ and R₉ together with the nitrogen atom to which they are attached form a 5- or 6-membered saturated or unsaturated heterocyclic ring optionally containing 1-2 further heteroatoms selected from oxygen, nitrogen or sulfur, the additional nitrogen being unsubstituted or substituted by alkyl substituted by halogen, hydroxyl or phenyl; and

R₇ is O or S;

Y is H, OH or NH₂;

Z₁, Z₂ and Z₃ each independently is O⁻ or BH₃⁻;

W₁ and W₂ each independently is O, CH₂, C(Hal)₂ or NH, wherein Hal is halogen, preferably F or Cl;

n is 0 or 1, provided that when n is 0 and W₂ is O, Z₁ is BH₃⁻; and when n is 1, at least one of W₁ and W₂ is not O;

m is 3 or 4; and

B⁺ represents a pharmaceutically acceptable cation,

and diastereoisomers thereof,

but excluding the compounds wherein n is 0, Z₁ and Z₃ are each O⁻, and W₂ is CH₂ or NH, and the compounds wherein n is 1 and Z₁ to Z₃ are each O⁻.

In another aspect, the present invention relates to a pharmaceutical composition comprising a compound of the general formula I but excluding the compounds wherein n is 0, Z₁ and Z₃ are each O⁻, and W₂ is CH₂ or NH, and the compounds wherein n is 1 and Z₁ to Z₃ are each O⁻, or a pharmaceutically acceptable salt thereof, and a pharmaceutically acceptable carrier or diluent.

The present invention further provides pharmaceutical compositions comprising a compound of the general formula I for treatment of a disease, disorder or condition modulated by P2Y receptors, such as type 2 diabetes or pain.

Thus, in a further aspect, the present invention relates to use of a compound of the general formula I or a pharmaceutically acceptable salt thereof for the preparation of a pharmaceutical composition for treatment of a disease, disorder or condition modulated by P2Y receptors.

In yet a further aspect, the present invention relates to a compound of the general formula I or a pharmaceutically acceptable salt thereof for treatment of a disease, disorder or condition modulated by P2Y receptors.

In still a further aspect, the present invention provides a method for treatment of a disease, disorder or condition modulated by P2Y receptors, such as type 2 diabetes or pain, in an individual in need, comprising administering to said individual an effective amount of a compound of the general formula I or a pharmaceutically acceptable salt thereof.

### BRIEF DESCRIPTION OF THE FIGURES

FIGS. 1A-1B show hydrolysis of the compound herein designated 2 under gastric juice-like conditions (in KCl/HCl buffer at pH 1.4 and 37° C.) as monitored by ³¹P NMR at 81 MHz. Changes of ³¹P NMR spectra of compound 2 as a function of time are shown in 1A; and determination of t_{1/2} of the above hydrolysis reaction, indicating t_{1/2} of 65 h, is shown in 1B.

FIGS. 2A-2C show hydrolysis of the compounds herein designated 3B and 4B under gastric juice-like conditions (in KCl/HCl buffer at pH 1.4 and 37° C.) as monitored by HPLC. FIGS. 2A and 2B show HPLC chromatograms of 3B at t=19 h and at t=71 h, respectively; and FIG. 2C shows determination of t_{1/2} of the above hydrolysis reactions, indicating t_{1/2} of 19 and 14.5 h, for 3B and 4B respectively.

FIG. 3 shows enzymatic hydrolysis of ATP, ADP and AMP in human blood serum at 37° C., as monitored by HPLC, indicating t_{1\2} of 3.6 h for ATP.

FIGS. 4A-4C show enzymatic hydrolysis of β,γ-CH₂-2MeS-ATP, 2, in human blood serum at 37° C., as monitored by HPLC. FIGS. 4A and 4B show HPLC chromatograms of the hydrolytic mixture in human blood serum at t=8 h and at t=15 h, respectively; and FIG. 4C shows determination of k (t_{1/2}) of the above hydrolysis reaction, indicating t_{1\2} of 12.7 h.

FIG. 5 shows that 2-MeS-adenosine-5′-O-(1-boranodiphosphate), 19, reduces glycemia following glucose challenge in rats. Starved Wistar rats (n=5) were treated intravenously (IV) with 2.5 mg/kg or saline 10 minutes after a glucose challenge, as described in Example 12. Glibenclamide (0.25 mg/kg) was given per os at −30 minutes as a positive control.

### DETAILED DESCRIPTION OF THE INVENTION

The present invention relates, in one aspect, to non-hydrolyzable nucleoside di- or triphosphate derivatives, which are P2Y receptor subtype selective agonists of the general formula I herein, as defined hereinabove.

As used herein, the term “halogen” includes fluoro, chloro, bromo, and iodo, and is preferably fluoro or chloro.

The term “hydrocarbyl” in any of the definitions of the different radicals R₁ to R₉ refers to a radical containing only carbon and hydrogen atoms that may be saturated or unsaturated, linear or branched, cyclic or acyclic, or aromatic, and includes C₁-C₈ alkyl, C₂-C₈ alkenyl, C₂-C₈ alkynyl, C₃-C₁₀ cycloalkyl, C₃-C₁₀ cycloalkenyl, C₆-C₁₄ aryl, (C₁-C₈)alkyl(C₆-C₁₄)aryl, and (C₆-C₁₄) aryl(C₁-C₈)alkyl.

The term “C₁-C₈ alkyl” typically means a straight or branched hydrocarbon radical having 1-8 carbon atoms and includes, for example, methyl, ethyl, n-propyl, isopropyl, n-butyl, sec-butyl, isobutyl, tert-butyl, n-pentyl, 2,2-dimethylpropyl, n-hexyl, n-heptyl, n-octyl, and the like. Preferred are C₁-C₆ alkyl groups, most preferably methyl. The terms “C₂-C₈ alkenyl” and “C₂-C₈ alkynyl” typically mean straight and branched hydrocarbon radicals having 2-8 carbon atoms and 1 double or triple bond, respectively, and include ethenyl, 3-buten-1-yl, 2-ethenylbutyl, 3-octen-1-yl, and the like, and propynyl, 2-butyn-1-yl, 3-pentyn-1-yl, and the like. C₂-C₆ alkenyl radicals are preferred. The term “C₃-C₁₀ cycloalkyl” means a cyclic or bicyclic hydrocarbyl group such as cyclopropyl, cyclobutyl, cyclopentyl, cyclohexyl, adamantyl, bicyclo[3.2.1]octyl, bicyclo[2.2.1]heptyl, and the like. The term “C₆-C₁₄ aryl” denotes a carbocyclic aromatic radical such as phenyl and naphthyl and the term “ar(C₁-C₈)alkyl” denotes an arylalkyl radical such as benzyl and phenetyl.

When the radical R₁ is a O-hydrocarbyl or S-hydrocarbyl radical or is hydrocarbyl substituted by a OR₄ or SR₄ radical, wherein R₄ is hydrocarbyl, each one of said hydrocarbyls is preferably a C₁-C₆ alkyl, most preferably methyl, or an aryl, most preferably phenyl, or an aralkyl, most preferably benzyl, radical.

When one or both of the radicals R₂ and R₃ are hydrocarbyls, each of these hydrocarbyls is preferably a C₁-C₆ alkyl, most preferably methyl, or an aryl, most preferably phenyl, or an aralkyl, most preferably benzyl, radical.

When the radical R₆ is a O-hydrocarbyl or S-hydrocarbyl radical or is hydrocarbyl substituted by a OR₈ or SR₈ radical, wherein R₈ is hydrocarbyl, each one of said hydrocarbyls is preferably a C₁-C₆ alkyl, most preferably methyl, or an aryl, most preferably phenyl, or an aralkyl, most preferably benzyl, radical.

In the group NR₄R₅, R₄ and R₅ each independently is H or hydrocarbyl as defined above or form together with the N atom to which they are attached a saturated or unsaturated, preferably a 5- or 6-membered, heterocyclic ring, optionally containing 1 or 2 further heteroatoms selected from nitrogen, oxygen, and sulfur. Such rings may be substituted, for example with one or two C₁-C₆ alkyl groups, or with one alkyl or hydroxyalkyl group at a second nitrogen atom of the ring, for example in a piperazine ring. Examples of radicals NR₄R₅ include, without being limited to, amino, dimethylamino, diethylamino, ethylmethylamino, phenylmethyl-amino, pyrrolidino, piperidino, tetrahydropyridino, piperazino, ethylpiperazino, hydroxyethylpiperazino, morpholino, thiomorpholino, thiazolino, and the like.

In the group NR₈R₉, R₈ and R₉ each independently is H or hydrocarbyl as defined above or form together with the N atom to which they are attached a saturated or unsaturated, preferably a 5- or 6-membered, heterocyclic ring, optionally containing 1 or 2 further heteroatoms selected from nitrogen, oxygen, and sulfur. Such rings may be substituted, for example with one or two C₁-C₆ alkyl groups, or with one alkyl or hydroxyalkyl group at a second nitrogen atom of the ring, for example in a piperazine ring. Examples of radicals NR₈R₉ include, without being limited to, amino, dimethylamino, diethylamino, ethylmethylamino, phenylmethyl-amino, pyrrolidino, piperidino, tetrahydropyridino, piperazino, ethylpiperazino, hydroxyethylpiperazino, morpholino, thiomorpholino, thiazolino, and the like.

The term “heteroaryl” refers to a radical derived from a mono- or poly-cyclic ring containing one to three heteroatoms selected from the group consisting of N, O and S, with unsaturation of aromatic character. Non-limiting examples of heteroaryl include pyrrolyl, furyl, thienyl, pyrazolyl, imidazolyl, oxazolyl, isoxazolyl thiazolyl, isothiazolyl, pyridyl, 1,3-benzodioxinyl, pyrazinyl, pyrimidinyl, 1,3,4-triazinyl, 1,2,3-triazinyl, 1,3,5-triazinyl, thiazinyl, quinolinyl, isoquinolinyl, benzofuryl, isobenzofuryl, indolyl, imidazo[1,2-a]pyridyl, pyrido[1,2-a]pyrimidinyl, benz-imidazolyl, benzthiazolyl, benzoxazolyl. The heteroaryl ring may be substituted. It is to be understood that when a polycyclic heteroaromatic ring is substituted, the substitution may be in the heteroring or in the carbocyclic ring.

The compounds described in the specification, both the compounds of formula I, the starting compounds and intermediates, and known compounds, are herein identified by the Arabic numbers 1-22 in bold. The full chemical structures are depicted in Appendix A, Schemes 1-5 herein. Compound 2 is also identified by the name β,γ-CH₂-2MeS-adenosine-5′-triphosphate, compound 3 is also identified by the name adenosine-β,γ-CH₂-5′-O-(1-boranotriphosphate), compound 4 is also identified by the name 2MeS-adenosine-β,γ-CH₂-5′-O-(1-boranotriphosphate), compound 17 is also identified by the name 2-MeS-adenosine-5′-dichloromethylene-diphosphate, compound 18 is also identified by the name 2-MeS-adenosine-5′-difluoromethylene-diphosphate, compound 19 is also identified by the name 2MeS-adenosine-5′-O-(Pα-borano) diphosphate, compound 20 is also identified by the name adenosine-β,γ-CCl₂-5′-O-(1-boranotriphosphate), compound is also identified by the name 2MeS-adenosine-β,γ-CCl₂-5′-O-(1-boranotriphosphate), and compound 22 is also identified by the name 2MeS-adenosine-β,γ-CF₂-5′-O-(1-boranotriphosphate).

In one embodiment, the compound of the present invention is a diphosphate derivative, wherein n is 0, that contain 0-2 BH₃⁻ groups. In preferred embodiments, the compound comprises no borano group; or it comprises a sole borano group at position α, wherein Z₁ is BH₃⁻ and Z₂ is O⁻, or at position β, wherein Z₃ is BH₃⁻ and Z₁ is O⁻; or two borano groups at positions α and β, namely, Z₁ and Z₃ are BH₃⁻.

In another embodiment, the compound of the present invention is a triphosphate derivative, namely n is 1, that contain 1-3 BH₃⁻ groups. In preferred embodiments, the compound comprises a sole borano group at position α, wherein Z₁ is BH₃⁻, and Z₂ and Z₃ are O⁻, at position β, wherein Z₂ is BH₃⁻, and Z₁ and Z₃ are O⁻, or at position γ, wherein Z₃ is BH₃⁻, and Z₁ and Z₂ are O⁻; two borano groups at positions α and β, wherein Z₁ and Z₂ are BH₃⁻, and Z₃ is O⁻, at positions α and γ, wherein Z₁ and Z₃ are BH₃⁻, and Z₂ is O⁻, or at positions β and γ, wherein Z₂ and Z₃ are BH₃⁻, and Z₁ is O⁻, or three borano groups at positions α, β and γ, wherein Z₁ to Z₃ are BH₃⁻.

In one embodiment, X is an adenine residue, namely, the compound of the present invention is an ATP or ADP derivative. Preferably, the compounds are those wherein X is an adenine residue, R₁ is H or S-alkyl, preferably S-methyl, R₂ and R₃ each independently is H; Y is OH; n is 1; Z₁ is BH₃⁻; Z₂ and Z₃ are O⁻; W₁ is O; and W₂ is CH₂, CF₂ or CCl₂; those wherein X is an adenine residue, R₁ is H or S-alkyl, preferably S-methyl, R₂ and R₃ each independently is H; Y is OH; n is 0; Z₁ and Z₃ are O⁻, and W₂ is CF₂ or CCl₂; and those wherein X is an adenine residue, R₁ is H or S-alkyl, preferably S-methyl, R₂ and R₃ each independently is H; Y is OH; n is 0; Z₁ is BH₃⁻; and W₂ is O.

In one preferred embodiment, the compound of the present invention is the compound of the general formula I wherein X is an adenine residue, R₁ is H, R₂ and R₃ are H, Y is OH, n is 1, Z₁ is BH₃⁻, Z₂ and Z₃ are O⁻, W₁ is O, and W₂ is CH₂ (compound 3). Due to the chiral center at Pα, this compound has a pair of two diastereoisomers (compounds 3A and 3B).

In another preferred embodiment, the compound of the present invention is the compound of the general formula I wherein X is an adenine residue, R₁ is SMe, R₂ and R₃ are H, Y is OH, n is 1, Z₁ is BH₃⁻, Z₂ and Z₃ are O⁻, W₁ is O, and W₂ is CH₂ (compound 4). More preferably, the compound of the present invention is the diastereoisomer B of compound 4, characterized by being the isomer with a retention time (Rt) of 5.57 min when separated from a mixture of diastereoisomers using a semi-preparative reverse-phase Gemini 5u column (C-18 110A, 250×10 mm, 5 micron), and isocratic elution [100 mM triethylammonium acetate (TEAA), pH 7 (A): MeOH (B), 85:15] with flow rate of 5 ml/min (compound 4B).

In a further preferred embodiment, the compound of the present invention is the compound of the general formula I wherein X is an adenine residue wherein R₁ is SMe, R₂ and R₃ are H, Y is OH, n is 0, Z₁ and Z₃ are O⁻, and W₂ is CCl₂ (compound 17).

In another preferred embodiment, the compound of the present invention is the compound of the general formula I wherein X is an adenine residue wherein R₁ is SMe, R₂ and R₃ are H, Y is OH, n is 0, Z₁ and Z₃ are O⁻, and W₂ is CF₂ (compound 18).

In still another embodiment, the compound of the present invention is the compound of the general formula I wherein X is an adenine residue wherein R₁ is SMe, R₂ and R₃ are H, Y is OH, n is 0, Z₁ is BH₃⁻, Z₃ is O⁻, and W₂ is O (compound 19). More preferably, the compound of the present invention is the diastereoisomer A of compound 19, characterized by being the isomer with a retention time (Rt) of 8.073 min when separated from a mixture of diastereoisomers using a semi-preparative reverse-phase Gemini 5u column (C-18 110A, 250×10 mm, 5 micron), and isocratic elution [100 mM TEAA, pH 7 (A): acetonitrile (B), 88:12] with flow rate of 1 ml/min (compound 19A).

In yet another preferred embodiment, the compound of the present invention is the compound of the general formula I wherein X is an adenine residue, R₁, R₂ and R₃ are H, Y is OH, n is 1, Z₁ is BH₃⁻, Z₂ and Z₃ are O⁻, W₁ is O; and W₂ is CCl₂ (compound 20). This compound has two diastereoisomers (compounds 20A and 20B).

In still a further preferred embodiment, the compound of the present invention is the compound of the general formula I wherein X is an adenine residue, R₁ is SMe, R₂ and R₃ are H, Y is OH, n is 1, Z₁ is BH₃⁻, Z₂ and Z₃ are O⁻, W₁ is O; and W₂ is CCl₂ (compound 21). This compound has two diastereoisomers (compounds 21A and 21B).

In yet a further preferred embodiment, the compound of the present invention is the compound of the general formula I wherein X is an adenine residue, R₁ is SMe, R₂ and R₃ are H, Y is OH, n is 1, Z₁ is BH₃⁻, Z₂ and Z₃ are O⁻, W₁ is O; and W₂ is CF₂ (compound 22). This compound has two diastereoisomers (compounds 22A and 22B).

In another embodiment, X is an uracil residue, namely, the compound of the present invention is a UTP or UDP derivative. Preferably, the compounds are those wherein X is an uracil residue, R₆ is H or S-alkyl, preferably S-methyl, R₇ is O or S; Y is OH; n is 1; Z₁ is BH₃⁻; Z₂ and Z₃ are O⁻; W₁ is O; and W₂ is CH₂, CF₂ or CCl₂; and those wherein X is an uracil residue, R₆ is H or S-alkyl, preferably S-methyl, R₇ is O or S; Y is OH; n is 0; Z₁ and Z₃ are O⁻; and W₂ is CF₂ or CCl₂.

The invention encompasses the compounds of formula I as defined above, the diastereoisomers thereof as well as pharmaceutically acceptable salts thereof.

In one embodiment, the cation B is an inorganic cation of an alkali metal such as, but not limited to, Na⁺, K⁺ and Li⁺.

In another embodiment, the cation B is ammonium (NH₄⁺) or it is an organic cation derived from an amine of the formula R₄N⁺, wherein each one of the Rs independently is selected from H, C₁-C₂₂, preferably C₁-C₆ alkyl, such as methyl, ethyl, propyl, isopropyl, butyl, and the like, phenyl, or heteroaryl such as pyridyl, imidazolyl, pyrimidinyl, and the like, or two of the Rs together with the nitrogen atom to which they are attached form a 3-7 membered ring optionally containing a further heteroatom selected from N, S and O, such as pyrrolydine, piperidine and morpholine.

In a further embodiment, the cation B is a cationic lipid or a mixture of cationic lipids. Cationic lipids are often mixed with neutral lipids prior to use as delivery agents. Neutral lipids include, but are not limited to, lecithins; phosphatidyl-ethanolamine; diacyl phosphatidylethanolamines such as dioleoyl phosphatidylethanolamine, dipalmitoyl phosphatidylethanolamine, palmitoyloleoyl phosphatidylethanolamine and distearoyl phosphatidylethanolamine; phosphatidyl-choline; diacyl phosphatidylcholines such as dioleoyl phosphatidylcholine, dipalmitoyl phosphatidylcholine, palmitoyloleoyl phosphatidylcholine and distearoyl phosphatidylcholine; fatty acid esters; glycerol esters; sphingolipids; cardiolipin; cerebrosides; ceramides; and mixtures thereof. Neutral lipids also include cholesterol and other 3β hydroxy-sterols.

Other neutral lipids contemplated herein include phosphatidylglycerol; diacyl phosphatidylglycerols such as dioleoyl phosphatidylglycerol, dipalmitoyl phosphatidylglycerol and distearoyl phosphatidylglycerol; phosphatidylserine; diacyl phosphatidylserines such as dioleoyl- or dipalmitoyl phosphatidylserine; and diphosphatidylglycerols.

Examples of cationic lipid compounds include, without being limited to, Lipofectin® (Life Technologies, Burlington, Ontario) (1:1 (w/w) formulation of the cationic lipid N-[1-(2,3-dioleyloxy)propyl]-N,N,N-trimethylammonium chloride and dioleoylphosphatidyl-ethanolamine); Lipofectamine™ (Life Technologies, Burlington, Ontario) (3:1 (w/w) formulation of polycationic lipid 2,3-dioleyloxy-N-[2(spermine-carboxamido)ethyl]-N,N-dimethyl-1-propanamin-iumtrifluoroacetate and dioleoylphosphatidyl-ethanolamine), Lipofectamine Plus (Life Technologies, Burlington, Ontario) (Lipofectamine and Plus reagent), Lipofectamine 2000 (Life Technologies, Burlington, Ontario) (Cationic lipid), Effectene (Qiagen, Mississauga, Ontario) (Non liposomal lipid formulation), Metafectene (Biontex, Munich, Germany) (Polycationic lipid), Eu-fectins (Promega Biosciences, San Luis Obispo, Calif.) (ethanolic cationic lipids numbers 1 through 12: C₅₂H₁₀₆N₆O₄.4CF₃CO₂H, C₈₈H₁₇₈N₈O₄S₂.4CF₃CO₂H, C₄₀H₈₄NO₃P.CF₃CO₂H, C₅₀H₁₀₃N₇O₃.4CF₃CO₂H, C₅₅H₁₁₆N₈O₂.6CF₃CO₂H, C₄₉H₁₀₂N₆O₃.4CF₃CO₂H, C₄₄H₈₉N₅O₃.2CF₃CO₂H, C₁₀₀H₂₀₆N₁₂O₄S₂.8CF₃CO₂H, C₁₆₂H₃₃₀N₂₂O₉.13CF₃CO₂H, C₄₃H₈₈N₄O₂.2CF₃CO₂H, C₄₃H₈₈N₄O₃.2CF₃CO₂H, C₄₁H₇₈NO₈P); Cytofectene (Bio-Rad, Hercules, Calif.) (mixture of a cationic lipid and a neutral lipid), GenePORTER® (Gene Therapy Systems, San Diego, Calif.) (formulation of a neutral lipid (Dope) and a cationic lipid) and FuGENE 6 (Roche Molecular Biochemicals, Indianapolis, Ind.) (Multi-component lipid based non-liposomal reagent).

Non-hydrolyzable nucleoside polyphosphate analogues have been used extensively as probes and inhibitors of nucleotide hydrolyzing enzymes (Labataille et al., 1995; Yanachkov et al., 1997; Spelta et al., 2003). Replacing a β,γ bridging oxygen in ATP with a methylene group (i.e., β,γ-CH₂-ATP) confers significant resistance to hydrolysis by nucleotide phosphohydrolases. For instance, β,γ-CH₂-ATP was identified as an inhibitor of glycerol kinase (Bystrom et al., 1997) and the 5′-β,γ-CF₂-TP moiety in 3′-azido-3′-deoxy-thymidine-5′-β,γ-CF₂-TP (AZT-5′-β,γ-CF₂-TP) rendered AZT, a potent inhibitor of human immunodeficiency-reverse transcriptase (HIV-RT), stable in serum and cell extracts (Wang et al., 2004). Likewise, β,γ-CH₂-ATP inhibited a detergent-solubilized e-NTPDase (Picher et al., 1996). Moreover, β,γ-CH₂-ATP selectively inhibited ATP hydrolysis catalyzed by e-NPP as well as e-NTPDase (Joseph et al., 2004).

β,γ-CH₂-ATP and analogues have been evaluated as metabolically stable ligands for certain P2 receptor subtypes (Spelta et al., 2003; El-Tayeb et al., 2005; Chen and Lin, 1997; Yegutkin and Burnstock, 2000; Zimmermann, 2000; Joseph et al., 2003). For instance, β,γ-CH₂-ATP was found to be a potent P2X₁R agonist (Burnstock et al., 1994; Janssens et al., 1996) but a weak agonist at P2X_{2/3}Rs (Spelta et al., 2003). β,γ-CH₂-ATP did not activate P2Y₁Rs (Burnstock et al., 1994; Janssens et al., 1996) but was a weak competitive antagonist at the P2Y₁R that inhibited responses elicited by 2-MeS-ADP (Sak et al., 2000).

Although hydrolytically stable in enzymatic assays, β,γ-CH₂-ATP was rapidly metabolized to adenosine in 1321N1 astrocytoma and C6 glioma cells by tightly coupled reactions involving serial catalysis by e-NPP (β,γ-CH₂-ATP->AMP) and CD73 (AMP->adenosine) (Joseph et al., 2004).

Although we were aware of the advantage of the β,γ-methylene group as a stabilizing isoster in β,γ-CH₂-ATP against NTPDase-mediated hydrolysis, we realized that it would not protect the labile α,β-phosphodiester bond. Furthermore, we suspected that this methylene isoster would reduce activity of the nucleotide at the P2Y₁R as mentioned above for β,γ-CH₂-ATP. Therefore, in addition to the β,γ-CH₂-group selected to protect this hydrolytically labile bond in ATP, the α phosphate was substituted by a boranophosphate moiety to stabilize the α,β-phosphodiester bond of ATP against hydrolysis by NTPDase (Nahum et al., 2002) and NPP (Nahum et al., 2006). In order to counteract the effect of the β,γ-methylene group and to enhance potency at P2Y₁R, we substituted the C2-position of ATP with a SMe group (Fischer et al., 1993).

Several chemical methods have been developed to form the pyrophosphonate bond in nucleotides. Nucleotide analogues in which β,γ-bridging oxygen is substituted by a methylene group are conventionally prepared via the activation of the 5′-phosphate of nucleoside-monophosphate (NMP) to form a phosphoryl donor followed by a reaction with methylene bisphosphonate salt (phosphoryl acceptor). Anhydrides of nucleoside-5′-monophosphates and methylene bisphosphonate were prepared by activation of NMP with carbonyl diimidazole (CDI) (Padyukova et al., 1999), trifluoroacetic anhydride and N-methylimidazole (Mohamady and Jakeman, 2005), or dicyclohexylcarbodiimide (DCC) (Myers et al., 1963) followed by condensation with methylene bisphosphonic acid or its salt.

2-MeS-β,γ-CH₂-ATP, 2, was previously obtained in a 3-step synthesis: first preparation of 2-MeS-AMP, then activation of the AMP analogue with carbonyl-diimidazole, and finally reaction with methylene-diphosphonic acid (Cusack et al., 1987). The conditions for these reactions and the product yields were not reported. Therefore, we attempted to improve the synthesis of this compound and to propose a short one-pot synthesis, as described in detail in Example 1 hereinafter and depicted in Scheme 1.

In order to ensure a selective reaction of 2-MeS-adenosine (Macfarlane, 1992) at 5′-OH, we used 2′,3′-methoxymethylidene-2-MeS-adenosine, 5a, as the starting material. Thus, 5a was first treated with POCl₃ in trimethylphosphate (TMP) in the presence of Proton Sponge™ (Aldrich) (1,8-bis(dimethylamino)naphthalene) at 0° C. for 3 h, followed by the addition of bis(tributylammonium)methylene-diphosphonate and tributylamine at 0° C. Finally, hydrolysis in 0.5 M TEAB and deprotection of the methoxymethylidene group generated 2 at a 9% overall yield.

The low overall yield of 2 encouraged us to use the non-protected nucleoside 5b as a starting material. Indeed, treatment of 5b with POCl₃ in TMP (in the presence of Proton Sponge™) at 0° C. for 2 h, followed by the addition of bis(tributylammonium)methylene-diphosphonate and tributylamine at 0° C. for 25 min, and hydrolysis in 0.5 M TEAB, yielded product 2 at a 20% overall yield. The major by-product was 2-MeS-AMP and no 2′,3′-cyclic-phosphate-2-MeS-β,γ-CH₂-ATP) was obtained (i.e., no signal was observed at +20 ppm), indicating that protection of the 2′,3′-hydroxyls is not necessary.

Previously, we have developed an efficient four-step-one-pot synthesis of analogue 1 (Nahum et al., 2002). Here, we have modified the synthesis for the preparation of 3 and 4, as described in detail in Examples 2-3 and depicted in Scheme 2. The use of phosphitylation and boronation reagents in the synthetic method requires the use of protected nucleoside starting materials. For this purpose, we have protected the nucleoside 2′,3′-hydroxyls with a methoxy-methylidene group, which remained stable throughout the entire synthesis and was efficiently removed in the last step.

The first synthetic step included phosphytilation of the 5′-OH of compound 9. For this purpose, we have tried several phosphitylation reagents. Thus, 9 was treated with [(iPr)₂N]₂PCl at 0° C. for several hours; however, most starting material was not consumed even after 14 h at RT. With chlorobenzodioxaphosphorine most starting material 9 was consumed after 15 min at RT. However, upon the addition of 1.5 eq methylene-bisphosphonate at RT for 10 min and 10 eq BH₃.SMe₂ at RT for 30 min, only traces of product 3 were obtained. Finally, PCl₃ was found to be the best phosphytilating agent. Starting material 9 was consumed in less than 30 min. Furthermore, due to the high reactivity of PCl₃, the coupling to methylene-bisphosphonate salt was rather rapid (11 min). Finally, BH₃.SMe₂ was added at 0° C. and then the reaction mixture was stirred at RT for 30 min. These conditions provided the product 3 at a 39% yield based on a 81 MHz ³¹P NMR of the crude reaction mixture. In addition to 3, AMP-α-BH₃ and adenosine-5′-H-phosphonate were obtained as by-products in a ratio of 1:0.46:˜1, respectively. These by-products were identified by both ³¹P NMR and MS (electron spray ionization).

Product 4 was obtained from 5a in the same way at a 28% overall yield after LC separation.

The identity and purity of the products were established by ¹H and ³¹P NMR, high-resolution fast atom bombardment (FAB) MS, and HPLC in two solvent systems. ³¹P NMR spectra of products 3 and 4 showed a typical Pα signal as a multiplet at about 83 ppm. ¹H NMR spectra of 3 and 4 showed borane hydrogen atoms as a very broad signal at about 0.4 ppm.

Due to the chiral center at Pα, analogues 3 and 4 are each obtained as a pair of two diastereoisomers. In both ¹H and ³¹P NMR spectra, there was a slight difference between the chemical shifts for the two diastereoisomers of 3 and 4. For instance, for 3 diastereoisomers, two sets of signals were observed for H8, at 8.59 and 8.56 ppm. These isomers were well-separated by reverse-phase HPLC with about 2 min difference in their retention times. The first eluting isomer was designated the A isomer, and the other was designated the β isomer.

In order to explore the suitability of the P2Y₁R agonists 2-4 as drug candidates, we evaluated their hydrolytic stability. In particular, the hydrolytic stability of β,γ-CH₂-ATP analogues 2-4 was monitored by either ³¹P NMR spectroscopy or HPLC-MS at conditions simulating gastric juice acidity, i.e., pH 1.4/37° C.

As shown in Examples 4-5 hereinafter, based on ³¹P NMR spectra, under these conditions, compound 2 exhibited relatively high stability and a pseudo first-order exponential decay rate equation with respect to its concentration, wherein its half life determined at pH 1.4/37° C. was 65 h. Similarly, based on HPLC, compound 3 (isomer B) exhibited a pseudo first-order exponential decay rate equation (the hydrolysis of compound 3 is depicted in Scheme 3). The half-life determined at pH 1.4/37° C. for compound 3B was 19 h. Likewise, half-life of compound 4 determined in the same way was 14.5 h. The hydrolysis rate constants of 2 and 3 represent ca. 3- to 11-fold improvement of their chemical stability as compared to that of 2-MeS-ATP-α-BH₃ under the same conditions (t_{1/2} of 5.9 h).

Previously, we have found that compound 1 was susceptible to hydrolysis by alkaline phosphatase degrading mostly to 2-MeS-AMP-α-BH₃, although small amounts of 2-MeS-ADP-α-BH₃ could be detected as well. Specifically, after incubating 1 in alkaline phosphatase for 12 min at 37° C., only 40% of 1 remained, whereas after 100 min, only traces of 1 could be detected by HPLC-MS.

Therefore, in order to compare the hydrolytic resistance of compounds 2-4 to alkaline phosphatase, to that of 1, we incubated the various analogues with the enzyme for 30 min at 37° C. As shown in Example 6, HPLC analysis of the enzymatic reaction mixture indicated that compounds 2-4 remained completely intact under these conditions.

The usage of nucleoside-5′-triphosphates for therapeutic purposes is limited due to their rapid dephosphorylation in extracellular media. The extracellular concentration of synthetic nucleotides is regulated by hydrolysis by ecto-ATPases (and synthesis by ecto-nucleotide diphosphokinases; see regulation of extracellular ATP) (Zimmermann, 2000; Yegutkin et al., 2001 and 2002; Lazarowski et al., 1997 and 2000). Four major families of ecto-nucleotidases have been identified, as described in Zimmermann (2000): (i) the ecto-nucleoside 5′-triphosphate diphosphohydrolases (e-NTPDases); (ii) the ecto-nucleotide pyrophosphatases (e-NPPs); (iii) the glycosylphosphatidylinositol (GPI)-anchored ecto-5′-nucleotidase; and (iv) the GPI-anchored alkaline phosphatase (APs). e-NTPDase1-3, which are cell surface enzymes, degrade extracellular ATP to ADP and ADP to AMP releasing inorganic phosphate, while e-NPP1-3 hydrolyze ATP directly to AMP and pyrophosphate. Extracellular AMP, in turn, can be degraded to adenosine by ecto-alkaline phosphatase. Blood serum contains dephosphorylating enzymes and therefore provides a good model system of the extracellular environment in vivo.

Phosphonate modified dNTP analogues displayed enhanced stability towards dephosphorylating enzymes in human blood serum (Arzumanov et al., 1996; Dyatkina et al., 1996; Shirokova and Dyatkina, 1996) and in muscle strips preparations (Cusack et al., 1987). Thus, in the latter preparation, no degradation of β,γ-CH₂-ATP and 2-MeS-β,γ-CH₂-ATP by ecto-nucleotidases was detected after 60 min incubation, during which time ATP was completely dephosphorylated (Cusack et al., 1987).

In order to determine the half-life of compounds 2-4 in human blood serum, these compounds were incubated in human blood serum and RPMI-1640 at 37° C. for 1 up to 144 h, and their hydrolysis was compared to that of ATP under the same conditions. As shown in Example 7, ATP was hydrolyzed to ADP and AMP with half-life of 3.6 h, while under the same conditions, compounds 2, 3A and 3B were mostly hydrolyzed to the corresponding nucleoside-monophosphate (boranophosphate) with half-lives of 12.7, 14.1 and 47.1 h, respectively. Using a different evaluation method, ATP was hydrolyzed with half-life of 7.7 h, while under the same conditions, compound 4B was hydrolyzed with half-life of 71.9 h. These values represent a 3.5-20-fold substitution-dependent enhancement of the metabolic stability of ATP.

In the experiment described in Example 11, the activity of various compounds of the formula I at the G protein-coupled P2YRs P2Y₁, P2Y₂, P2Y₄ and P2Y₆, expressed in human astrocytoma cells, was examined. The compounds that were first examined were compounds 2-4 and as shown, compounds 2 and 4B were agonists of the P2Y₁R with EC₅₀'s of 0.08 and 17.2 μM, respectively, as compared to 0.004 μM for 2-MeS-ADP, and had a slight agonistic effect at 100 μM on P2Y₆R. Compounds 3A, 3B and 4A had insignificant activities at the P2YRs tested.

Although compound 2 was found to be more potent and selective P2Y₁R agonist compared to compound 4B, it was about one order of magnitude less potent than 2-MeS-ADP (EC₅₀ 4 nM) or 2-MeS-ATP in a related system (EC₅₀ 1 nM in HEK293 cells expressing rP2Y₁R. EC₅₀ was determined by Ca²⁺ mobilization) (Major et al., 2004). The relatively reduced potency of 2 may be related to the higher pKₐ value of phosphonate vs. phosphate (8.4 vs. 6.5) (Blackburn et al., 1981). In particular, whereas under the assay conditions, pH 7.4 (and possibly within the receptor binding-pocket), 91% of 2-MeS-ADP(ATP) is ionized, compound 2 (phosphonate moieties) is only 9% ionized. The result of this low degree of ionization of 2 is weaker interactions with the receptor as described below. In order to evaluate whether this hypothesis is correct, we have then examined the agonistic effect of 2-MeS-ADP-α,β-CCl₂ (or CF₂) (17 and 18, respectively), 2-MeS-ADPαB (isomer 19A) and 2-MeS-ATPαB-β,γ-CCl₂ (isomers 21A and 21B) at the P2Y₁R. The pKₐ value of the terminal phosphonate in compounds 17, 18 and 21, and in particular, in the CF₂ analogues, is about 6.7, suggesting that these analogues should have improved interactions with, thus significantly improved activity at P2Y₁ receptors. Nevertheless, as further shown in Example 11, while each one of these compounds was found to be less potent and selective P2Y₁R agonist compared to compound 2, it was compound 19A that was found to be the most potent and selective to P2Y₁R (EC₅₀'s of 3.1, 0.98, 0.038, 0.57 and 1.2 μM for compounds 17, 18, 19A, 21A and 21B, respectively).

Previously we have calculated a model of the 2-BuS-ATP:P2Y₁R complex and found that Pβ,γ of this nucleotide analogue interacts with positively charged Lys240 and Arg128 within the P2Y₁R binding-site (Major and Fischer, 2004). Thus, we assume that the higher pKₐ of the phosphonate moieties of compound 2 may result in the loss of important ionic interactions with the P2Y₁R binding pocket, and consequently reduced EC₅₀ values.

Although geometrical considerations due to differences in the PCP vs. POP angle and C—P vs. O—P bond length may also play a role in the molecular recognition of 2 vs. 2-MeS-ADP(ATP), still these differences are rather small (PCP and POP angles—117.0 and 128.7; and C—P and O—P bond lengths—1.79 and 1.63 Å, respectively), suggesting that the major parameter determining the affinity and activity of 2 is the pKₐ value of the phosphonate group.

The fact that compound 2, 4B, 17, 18, 19A, 21A and 21B were active at P2Y₁R whereas compounds 3A and 3B were practically inactive may be due to improved interactions of 2-MeS-adenine moiety vs. adenine with the P2Y₁R binding-pocket (Major et al., 2004, Major and Fischer, 2004). In particular, in addition to the strong recognition network observed for the triphosphate moiety of ATP analogues at P2Y₁R, another important network of interactions, although weaker, was observed for the adenine ring (Major and Fischer, 2004). These interactions are important as they improve affinity to the receptor and determine the receptor sub-type selectivity. Specific H-bonding interactions to N1, N⁶, and N7 are provided by Arg310, Ser314 and possibly Tyr58. These interactions are enhanced in the presence of SMe group at C2, due to electronic effects. Namely, the thiomethyl group increases the electron density at the adenine N1-position, thus increasing its potency as H-bonding acceptor. In addition, π-stacking interaction of the adenine ring with Phe131 is further enhanced upon substitution of C2 with a SMe group, as in this derivative the adenine ring functions as a charge donor molecule in a π-stacking charge transfer complex. Moreover, this substituent yields a more rigid fit between the adenine moiety and the receptor. Specifically, C2-thiomethyl group at compounds 2, 4B, 17, 18, 19A, 21A and 21B forms hydrophobic interactions with the P2Y₁R hydrophobic pocket involving Leu104, Pro105, Ile130 and Leu135.

We have reported that C2-substituted ATP-α-B analogues are not well tolerated by the P2Y₂R (Tulapurkar et al., 2004). 2-C1- and 2-MeS-ATP-α-B were found very weak agonists at the P2Y₂R. In view of that, our findings here for the inactivity of phosphonates 2 and 4A at P2Y₂R are consistent with these earlier reports. Inactivity of compounds 2-4 at the P2Y_{4/6}-Rs was expected as these receptors are selective for uridine nucleotide agonists.

In summary, as the relative potency of ATP or ADP analogues is usually related to their resistance to hydrolysis (Adams, 1994; Burnstock and Kennedy, 1985; Evans and Kennedy, 1994), we have developed novel non-hydrolyzable P2Y₁R agonists. The EC₅₀ values of compounds 2, 4B, 17, 18, 19A, 21A and 21B are in the range of 1 to 3 orders of magnitude higher than those of the corresponding phosphate analogues, 2-MeS-ATP and 1A (Nahum et al., 2002; Major et al., 2004). Yet, the great advantage of the former analogues is their significantly higher survival at human blood serum and in the drastic conditions of gastric juice. These features make these analogues attractive and selective therapeutic candidates for health disorders involving the P2Y₁R.

In another aspect, the present invention relates to a pharmaceutical composition comprising a compound of the general formula I but excluding the compounds wherein n is 0, Z₁ and Z₃ are each O⁻, and W₂ is CH₂ or NH, and the compounds wherein n is 1 and Z₁ to Z₃ are each O⁻, or a pharmaceutically acceptable salts thereof, and a pharmaceutically acceptable carrier or diluent.

In a further aspect, the present invention provides pharmaceutical compositions comprising a compound of the general formula I for treatment of a disease, disorder or condition modulated by P2Y receptors. Preferred compounds for such uses include compounds 2, 4, more preferably 4B, 17, 18, 19, more preferably 19A, 21A and 21B, or pharmaceutically acceptable salts thereof.

The disease or disorder modulated by P2Y receptors may be cancer, a disorder associated with platelet aggregation, a cardiovascular disease or disorder, a disease associated with a disorder of mucous hydration, secretion and clearance, or type 2 diabetes.

The types of cancer that can be treated by the compound of the general formula I may be, without being limited to, leukemia, lymphoma, multiple myeloma, melanoma, prostate, brain, colon, ovarian, breast, skin, lung, esophagus and bladder cancers.

The cardiovascular disease or disorder may be, without being limited to, ischemia/reperfusion injury, myocardial infarction, and long-standing heart failure.

The diseases associated with a disorder of mucous hydration, secretion and clearance include, without being limited to, chronic obstructive pulmonary disease, pneumonia, bronchitis, cystic fibrosis, primary ciliary dyskinesia, sinusitis, otitis media, dry eye disease, glaucoma, nasolacrimal duct obstruction, edematous retinal disorders, retinal degeneration, vaginal dryness, dry mouth, gastroesophaphageal reflux, and constipation.

As disclosed in the aforesaid WO 03/034978, selective P2Y₁R agonists based on boranophosphate isosters of ATP analogues were found to be highly potent insulin secretagogues at perfused rat pancreas, wherein the most effective agonist was 2-MeS-ATP-α-B, 1, which induced a 9-fold enhancement of insulin secretion as compared to basal secretion with an EC₅₀ of 28 nM. Thus, pharmaceutical compositions comprising compounds of the general formula I, which are highly selective P2Y₁R agonists, preferably compounds 2, 4B, 17, 18, 19A, 21A and 21B can be used as insulin secretagogues for treatment of type 2 diabetes.

Thus, in a preferred embodiment, the disease or disorder modulated by P2Y receptors is type 2 diabetes.

Since pain is at least partially modulated by P2Y₁ receptors as well, pharmaceutical compositions comprising the compound of the general formula I can further be used for pain control.

The pharmaceutical compositions containing a compound of the general formula I may be prepared by conventional techniques, e.g. as described in Remington: The Science and Practice of Pharmacy, 19th Ed., 1995. The compositions may appear in conventional forms, for example capsules, tablets, solutions or suspensions, emulsion, cream, spray and the like.

The route of administration may be any route which effectively transports the active compound to the appropriate or desired site of action, the oral route being preferred. If a solid carrier is used for oral administration, the preparation may be tableted, placed in a hard gelatin capsule in powder or pellet form or it can be in the form of a lozenge. If a liquid carrier is used, the preparation may be in the form of a syrup, emulsion or soft gelatin capsule. Tablets, dragees, or capsules having talc and/or a carbohydrate carrier or binder or the like are particularly suitable for oral application. Preferable carriers for tablets, dragees, or capsules include lactose, corn starch, and/or potato starch.

In a further aspect, the present invention provides a method for treatment of a disease, disorder or condition modulated by P2Y receptors, such as type 2 diabetes or pain, in an individual in need, comprising administering to said individual an effective amount of a compound of the general formula I or a pharmaceutically acceptable salt thereof.

The invention will now be illustrated by the following non-limiting Examples.

### EXAMPLES

### Experimental

### General

All air and moisture sensitive reactions were carried out in flame-dried, argon flushed, two neck flasks sealed with rubber septa, and the reagents were introduced with a syringe. Progress of reactions was monitored by TLC on precoated Merck silica gel plates (60E-254). Visualization was accomplished by UV light. Compounds were characterized by nuclear magnetic resonance using Bruker DPX-300, DMX-600 or AC-200 spectrometers. ¹H NMR spectra were measured at 200, 300 or 600 MHz. Nucleotides were characterized also by ³¹P NMR in D₂O, using 85% H₃PO₄ as an external reference on Bruker AC-200 and DMX-600 spectrometers. High resolution mass spectra were recorded on an AutoSpec-E FISION VG mass spectrometer by chemical ionization. Nucleotides were analyzed under ESI (electron spray ionization) on a Q-TOF micro-instrument (Waters, UK). Primary purification of the nucleotides was achieved on an LC (Isco UA-6) system using a column of Sephadex DEAE-A25, swollen in 1 M NaHCO₃ at 4° C. for 1 day. The resin was washed with deionized water before use. The LC separation was monitored by UV detection at 280 nm. A buffer gradient 0-0.8 M NH₄HCO₃ (500 ml water:500 ml buffer) was applied. Final purification of the nucleotides and separation of the diastereomeric pairs were achieved on a HPLC (Merck-Hitachi) system using a semi-preparative reverse-phase column (Gemini 5u C-18 110A, 250×10.00 mm, 5 micron, Phenomenex, Torrance, USA). The purity of the nucleotides was evaluated on an analytical reverse-phase column system (Gemini 5u, C-18, 110A, 150×4.60 mm, 5 micron, Phenomenex, Torrance, Calif., USA), in two solvent systems as described below.

All commercial reagents were used without further purification, unless otherwise noted. All reactants in moisture sensitive reactions were dried overnight in a vacuum oven. RPMI (Roswell Park Memorial Institute) 1640 buffer was obtained from Sigma-Aldrich. 2′,3′-O-Methoxymethylidene adenosine derivatives were prepared as described by Nahum et al. (2002). 2′,3′-O-Methoxymethylidene-2-MeS-adenosine was separated on a MPLC system (Biotage, Kungsgatan, Uppsala, Sweden) using a silica gel column (25+M column) and the following gradient scheme: 3 column volumes (CV) of 100:0 (A) CHCl₃ (A):(B) EtOH, 5 CV of a gradient from 100:0 to 90:10 of A:B and 4 CV of 90:10 A:B at a flow rate of 12.5 ml/min. Evaluation of chemical stability and pH measurements were performed with an Orion microcombination pH electrode and a Hanna Instruments pH meter.

### Intracellular Calcium Measurement

Human 1321N1 astrocytoma cells stably expressing the turkey P2Y₁, human P2Y₂, human P2Y₄ or rat P2Y₆ were grown in Dulbecco's modified Eagle's medium containing 5% (v/v) fetal bovine serum, 100 units/ml penicillin, 100 μg/ml streptomycin and 500 μg/ml Geneticin (G-418, Life Technologies, Inc). Changes in the intracellular free calcium concentration, [Ca²⁺]ᵢ were detected by dual-excitation spectrofluorometric analysis of cell suspensions loaded with fura-2, as previously described (Garrad et al., 1998; Grynkiewicz et al., 1985). Cells were assayed in 10 mM Hepes-buffered saline (pH 7.4) containing 1 mM CaCl₂ and 1 mM MgCl₂. The cells were pelleted in a microfuge and resuspended in 2 ml of buffer. Concentration-response data were analyzed with the Prism curve fitting program (GraphPAD Software, San Diego, Calif.). Three experiments were conducted on separate days for each P2Y receptor subtype.

### Example 1

### β,γ-CH₂-2MeS-adenosine-5′-triphosphate, 2, synthesis

β,γ-CH₂-2MeS-adenosine-5′-triphosphate, 2, was prepared by the two methods as depicted in Scheme 1 and described hereinbelow:

Method A. Bis(tributylammonium)methylene diphosphonate salt was prepared as described above. 1,8-bis(dimethylamino)naphthalene (117 mg, 0.57 mmol, 1.5 eq) was added at 0° C. to 2′,3′-O-methoxymethylidene-2-MeS-adenosine, 5a, (130 mg, 0.37 mmol) in trimethylphosphate (2 ml) in a flame-dried two-neck flask under N₂, and the reaction was stirred for 20 min until a clear solution was attained. POCl₃ (67 μl, 1.09 mmol, 3 eq) was added at 0° C. The solution was stirred at 0° C. for 3 h. A 0.5 M solution of bis(tributylammonium)methylene diphosphonate salt (386 mg, 2.19 mmol, 6 eq) in dry DMF (4.3 ml) and tributylamine (360 μl, 1.46 mmol, 4 eq) were added at 0° C. and the reaction mixture was stirred for 1.6 min. A 0.25 M solution of ammonium acetate (10 ml) was added at room temperature and the reaction mixture was stirred for 30 min, and then freeze-dried. The resulting residue was applied to an activated Sephadex DEAE-A25 column (0-0.8 M NH₄HCO₃, total volume of 1 l). The relevant fractions were collected and freeze-dried, and excess NH₄HCO₃ was removed by repeated freeze-drying with deionized water to yield product 8a as a white solid. Product 5 was treated with 18% HCl solution until pH 2.3 was attained, and then stirred for 3 h at room temperature. Finally, the mixture was treated with 24% NH₄OH solution and pH was adjusted to 9. The solution was stirred for 45 min, and then freeze-dried. The residue was separated on an HPLC column to obtain pure 2. The separation was accomplished using a semi-preparative reverse-phase Gemini 5u C-18 110A column (250×10.00 mm, 5 micron) and isocratic elution using Solvent System I, by applying 85:15 of (A) 100 mM triethylammonium acetate (TEAA), pH 7 to (B) MeOH, at a flow rate of 5 ml/min. The relevant fractions (Rt=12.09 min) were freeze-dried. The excess buffer was removed by repeated freeze-drying cycles, and the solid residue was dissolved each time in deionized water. Finally, the nucleotide triethylammonium counter ions were exchanged for Na⁺ ions by passing the pure product 1 through a Sephadex-CM C-25 Na⁺-form column. Product 2 was obtained in 10% (23 mg) yield after LC separation. Retention time on a semi-preparative column: 12.09 min. The spectral data for 2 were consistent with that described by Mohamady and Jakeman (2005).

Method B. Bis(tributylammonium)methylene diphosphonate salt was prepared as described above. 1,8-bis(dimethylamino)naphthalene (41 mg, 0.19 mmol, 2 eq) was added at 0° C. to 2-MeS-adenosine, 5b, (30 mg, 0.09 mmol) in trimethylphosphate (1 ml) in a flame-dried two-neck flask under N₂, and the reaction was stirred for 20 min until a clear solution was attained. POCl₃ (26 μl, 0.28 mmol, 3 eq) was added at 0° C. The solution was stirred at 0° C. for 2 h. A 1 M solution of bis(tributylammonium) methylene diphosphonate salt (101 mg, 0.57 mmol, 6 eq) in dry DMF (480 μl) and tributylamine (91 μl, 0.38 mmol, 4 eq) was added at 0° C. and the reaction mixture was stirred for 1.6 min. Then, 0.5 M triethylammonium bicarbonate (TEAB) solution (10 ml) was added at room temperature and the reaction mixture was stirred for 30 min, and then freeze-dried. The resulting residue was applied to an activated Sephadex DEAE-A25 column (0-0.8 M NH₄HCO₃, total volume of 1 l). The relevant fractions were collected and freeze-dried, and excess NH₄HCO₃ was removed by repeated freeze-drying with deionized water to yield product 2 as a white solid. The residue was separated on an HPLC column to obtain pure 2. The separation was accomplished using a semi-preparative reverse-phase Gemini 5u C-18 110A column (250×10.00 mm, 5 micron) and Solvent System I (see hereinabove) with a gradient from (92:8 to 70:30 A:B) over 20 min at a flow rate of 5 ml/min. The relevant fractions (Rt=11.94 min) were freeze-dried. The excess buffer was removed by repeated freeze-drying cycles, and solid residue was dissolved each time in deionized water. Finally, the nucleotide triethylammonium counter ions were exchanged for Na⁺ ions by passing the pure product 1 through a Sephadex-CM C-25 Na⁺-form column. Product 2 was obtained in 10% (11 mg) yield after LC separation. The spectral data for 2 were consistent with that described by Mohamady and Jakeman (2005).

### Example 2

### Adenosine-β,γ-CH₂-5′-O-(1-boranotriphosphate), 3, synthesis, separation and characterization

Adenosine-β,γ-CH₂-5′-O-(1-boranotriphosphate), 3, synthesis

Bis(tributylammonium)methylene diphosphonate salt was prepared by the addition of Bu₃N (2 eq) to methylene diphosphonic free acid in EtOH and stirring for 2 h at room temperature followed by solvent removal under reduced pressure to give a white solid. As depicted in Scheme 2, 2′,3′-O-methoxymethylidene adenosine, 9, (100 mg, 0.32 mmol) was dissolved in trimethylphosphate (2.5 ml) in a flame-dried two-neck flask under N₂. 1,8-bis(dimethylamino)naphthalene (138 mg, 0.65 mmol, 2 eq) was added at 0° C. and the reaction was stirred for 20 min until a clear solution was attained. PCl₃ (56 μl, 0.65 mmol, 2 eq) was added at 0° C., and a white solid precipitated. The suspension was stirred at 0° C. for 30 min. Then, a 1 M solution of bis(tributylammonium)methylene diphosphonate salt (642 mg, 1.94 mmol, 6 eq) in dry DMF (1.8 m) and tributylamine (308 μl, 1.29 mmol, 4 eq) were added at 0° C. and the reaction mixture was stirred for 11 min. A 2 M solution of BH₃.SMe₂ complex in THF (2.2 ml, 3.9 mmol, 10 eq) was added at 0° C., and the reaction mixture became clear. The solution was stirred for 5 min at 0° C. and then for 30 min at room temperature. Finally, a 0.5 M TEAB solution (10 ml) was added at room temperature and the mixture was stirred for 60 min, and then freeze-dried. The resulting residue was applied to an activated Sephadex DEAE-A25 column (0-0.8 M NH₄HCO₃, total volume of 1 l). The relevant fractions were collected and freeze dried, and excess NH₄HCO₃ was removed by repeated freeze-drying cycles with deionized water. Product 13a was obtained as a white solid. Product 13a was treated with 18% HCl until pH 2.3 was attained, and then stirred for 3 h at room temperature. Finally, the mixture was treated with 24% NH₄OH solution, and pH was adjusted to 9. The solution was stirred for 45 min at room temperature and then freeze-dried. The diastereomeric pair of product 3 was separated on an HPLC column under the conditions described below. Finally, purified isomers 3A and 3B were passed through a Sephadex-CM C-25 Na⁺-form column to exchange triethylammonium counter ions for Na⁺ ions.

Adenosine-β,γ-CH₂-5′-O-(1-boranotriphosphate), 3, separation

The separation of the diastereomeric pair of 3 was accomplished using a semi-preparative reverse-phase Gemini 5u column (C-18 110A, 250×10.00 mm, 5 micron) and isocratic elution using Solvent System I (see Example 1) at 89:11 A:B at a flow rate of 5 ml/min, followed by a final separation of the two diastereoisomers using an analytical Gemini 5u column (C-18 110A, 150×4.60 mm) by applying Solvent System I (see Example 1) with a gradient from 90:10 to 70:30 A:B over 20 min at a flow rate of 1 ml/min. Fractions containing the same isomer [Rt=6.33 min (isomer A), 7.73 min (isomer B)] were collected and freeze-dried. The excess buffer was removed by repeated freeze-drying cycles with the solid residue dissolved each time in deionized water. Diastereoisomers 3A and 3B were obtained in 36% (66 mg) overall yield after LC separation.

Adenosine-β,γ-CH₂-5′-O-(1-boranotriphosphate), 3A, characterization

Retention time on a semi-preparative column: 7.64 min. ¹H NMR (D₂O, 600 MHz): δ 8.59 (s, H-8, 1H), 8.25 (s, H-2, 1H), 6.14 (d, J=4.8 Hz, H-1′, 1H), (H2′ signal is hidden by the water signal at 4.78 ppm), 4.60 (m, H-3′, 1H), 4.39 (m, H-4′, 1H), 4.27 (m, H-5′, 1H), 4.14 (m, H-5″, 1H), 2.25 (t, J=20.4 Hz, CH₂, 2H), 0.37 (m, BH₃, 3H) ppm. ³¹P NMR (D₂O, 600 MHz): δ 82.81 (m, P_{α}-BH₃), 13.92 (s, P_{γ}), 11.22 (br s, P_{β}) ppm. MS-ESI m/z: 502 (M⁻). TLC (NH₄OH:H₂O:isopropanol 2:8:11), R_{f}=0.23. Purity data obtained on an analytic column: Retention time: 3.55 min (100% purity) using Solvent System I (see Example 1) with a gradient from 90:10 to 70:30 A:B over 10 min at a flow rate of 1 ml/min). Retention time: 2.53 min (95.5% purity) using Solvent System II, a gradient from 90:10 to 80:20 of (A) 0.01 M KH₂PO₄, pH=4.5 to (B) MeOH over 10 min at a flow rate of 1 ml/min).

Adenosine-β,γ-CH₂-5′-O-(1-boranotriphosphate), 3B, characterization

Retention time on a semi-preparative column: 9.67 min. ¹H NMR (D₂O, 300 MHz): δ 8.56 (s, H-8, 1H), 8.24 (s, H-2, 1H), 6.14 (d, J=5.1 Hz, H-1′, 1H), (H2′ signal is hidden by the water signal at 4.78 ppm), 4.52 (m, H-3′, 1H), 4.39 (m, H-4′, 1H), 4.23 (m, H-5′, 1H), 4.17 (m, H-5″, 1H), 2.30 (t, J=20.10 Hz, CH₂, 2H), 0.40 (m, BH_{3J} 3H) ppm. ³¹P NMR (D₂O, 600 MHz): δ 82.50 (m, P_{α}-BH₃), 14.10 (s, P_{γ}), 11.03 (br s, P_{β}) ppm. MS-ESI m/z: 502 (M⁻). TLC (NH₄OH:H₂O:isopropanol 2:8:11), R_{f}=0.23. Purity data obtained on an analytic column: Retention time: 4.09 min (92.6% purity) using Solvent System I (see Example 1) with a gradient from 90:10 to 70:30 A:B over 10 min at a flow rate of 1 ml/min). Retention time: 3.66 min (95.5% purity) using Solvent System II (see hereinabove) with a gradient from 95:10 to 80:20 A:B over 10 min at a flow rate of 1 ml/min).

### Example 3

2MeS-adenosine-β,γ-CH₂-5′-O-(1-boranotriphosphate), 4, synthesis, separation and characterization

2MeS-adenosine-β,γ-CH₂-5′-O-(1-boranotriphosphate), 4, synthesis

Product 4 was obtained from 5a in the same way as described in Example 2 for product 3 and depicted in Scheme 2 hereinafter, at a 28% overall yield after LC separation.

2MeS-adenosine-β,γ-CH₂-5′-O-(1-boranotriphosphate), 4, separation

The separation of 4 diastereoisomers was accomplished using a semi-preparative reverse-phase Gemini 5u column (C-18 110A, 250×10.00 mm, 5 micron), and isocratic elution by applying Solvent System I (see Example 1) at 75:25 A:B at a flow rate of 5 ml/min. Final separation of the two diastereoisomers was achieved using an analytical Gemini 5u column (C-18 110A, 150×4.6 mm) and Solvent System I (see Example 1) with a gradient from 82:18 to 74:26 A:B over 20 min at a flow rate of 1 ml/min. Fractions containing the same isomer [Rt=9.79 min (isomer A), 11.53 min (isomer B)] were collected and freeze-dried. The excess buffer was removed by repeated freeze-drying cycles with the solid residue dissolved each time in deionized water. Diastereoisomers 4A and 4B were obtained in 28% (38 mg) overall yield after LC separation.

2MeS-adenosine-β,γ-CH₂-5′-O-(1-boranotriphosphate), 4A, characterization.

Retention time on a semi-preparative column: 5.29 min. ¹H NMR (D₂O, 600 MHz): δ 8.30 (s, H-8, 1H), 6.12 (d, J=4.98 Hz, H-1′, 1H), (H2′ signal is hidden by the water signal at 4.78 ppm), 4.50 (m, H-3′, 1H), 4.25 (m, H-4′, 1H), 4.14 (m, H-5′, 1H), 4.05 (m, H-5″, 1H), 2.95 (s, CH₃, 3H), 2.17 (t, J=20.10 Hz, CH₂, 2H), 0.42 (m, BH₃, 3H) ppm. ³¹P NMR (D₂O, 600 MHz): δ 83.60 (m, P_{α}-BH₃), 14.61 (s, P_{γ}), 10.26 (br s, P_{β}) ppm. MS-ES m/z: 548 (M). TLC (NH₄OH:H₂O:isopropanol 2:8:11), R_{f}=0.44. Purity data obtained on an analytic column: Retention time: 4.24 min (94.3% purity) using Solvent System I (see Example 1) with a gradient from 80:20 to 60:40 A:B over 10 min at a flow rate of 1 ml/min). Retention time: 2.99 min (99.5% purity) using Solvent System II (see Example 2) with a gradient from 75:25 to 65:35 A:B over 10 min at a flow rate of 1 ml/min).

2MeS-adenosine-β,γ-CH₂-5′-O-(1-boranotriphosphate), 4B, characterization

Retention time on a semi-preparative column: 5.57 min. ¹H NMR (D₂O, 600 MHz): δ 8.29 (s, H-8, 1H), 6.99 (m, H-1′, 1H), (H2′ signal is hidden by the water signal at 4.78 ppm), 4.47 (m, H-3′, 1H), 4.27 (m, H-4′, 1H), 4.15 (m, H-5′, 1H), 4.08 (m, H-5″, 1H), 2.49 (s, CH₃, 3H) 2.18 (t, J=19.20 Hz, CH₂, 2H), 0.32 (m, BH₃, 3H) ppm. ³¹P NMR (D₂O, 600 MHz): δ 84.13 (m, P_{α}-BH₃), 14.85 (s, P_{γ}), 10.04 (br s, P_{β}) ppm. MS-ESI m/z: 548 (M⁻). TLC (NH₄OH:H₂O:isopropanol 2:8:11), R_{f}=0.44. Retention time: 2.12 min (94% purity) using a gradient of (A) 100 mM TEAA, pH 7 to (B) CH₃CN from 70:30 to 40:60 A:B over 10 min at a flow rate of 1 ml/min). Retention time: 1.38 min (100% purity) using Solvent System II (see Example 2) with a gradient from 50:50 to 40:60 A:B over 10 min at a flow rate of 1 ml/min).

### Example 4

### The Chemical Stability of Compound 2 Evaluated by ³¹P NMR

The stability of 2 at pH 1.4 and 37° C. was evaluated by ³¹P NMR to monitor possible dephosphorylation products. Compound 2 (1.5 mg) was dissolved in 0.2 M HCl/KCl (0.35 ml) and D₂O (40 μl). The final pH was adjusted to 1.4 by adding 0.2 M HCl (20 μl). The solution was kept in an oil bath at 37° C. Spectra were recorded at 12 h time intervals for 11 days. The number of scans in every experiment was 500. The percentage of phosphate ester hydrolysis is based on integration of the P_{α} signal of β,γ-CH₂-2MeS-ATP (−10.5 ppm) and the P_{α} signal of the hydrolysis product 2MeS-AMP, 9, (0.7 ppm). The hydrolysis rate was determined by measuring the change in the integration of the respective NMR signals with time.

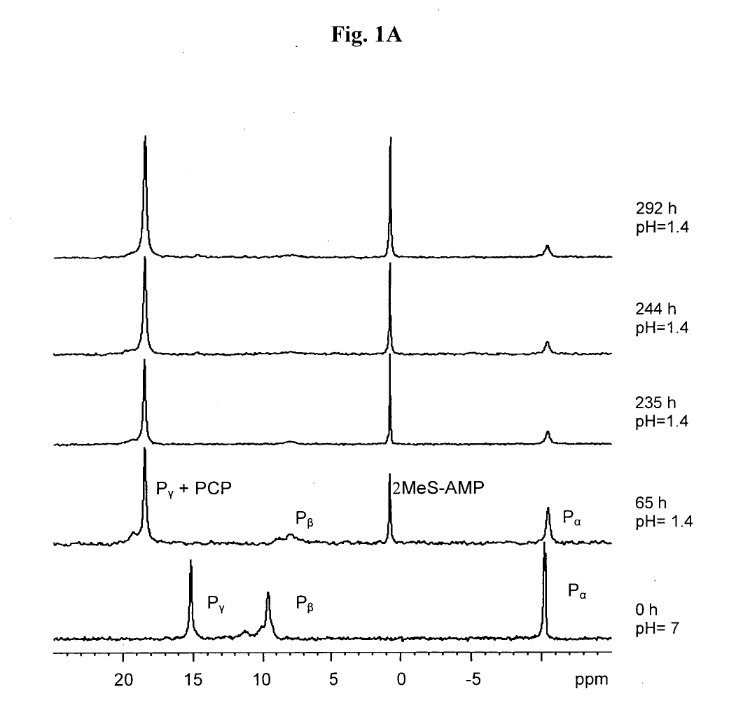

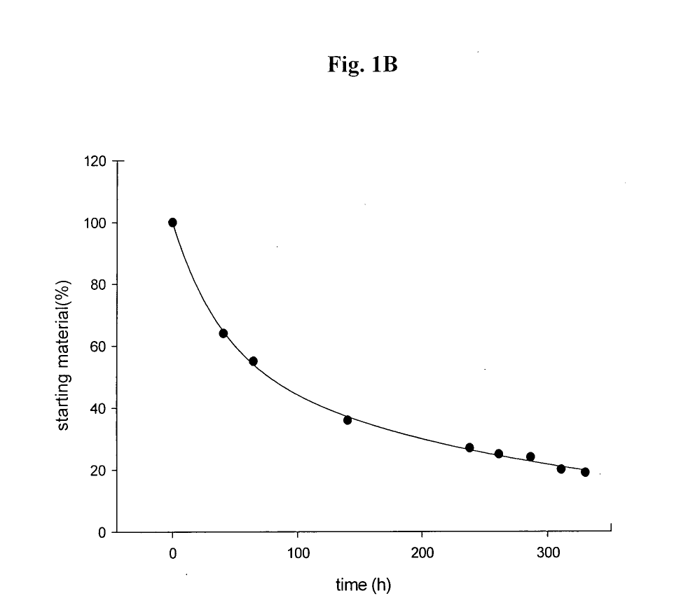

As shown in FIG. 1A, under these conditions compound 2 exhibited relatively high stability. In particular, in addition to starting material 2, increasing amounts of 2-MeS-AMP were observed with time. Thus, the signal at 0 ppm (Pα of 2-MeS-AMP) has gradually emerged, whereas the signal at −11 ppm (Pα of 2) has decreased with time. The intensity changes of the ³¹P NMR signal of the Pα of 2-MeS-AMP (as a percentage of total Pα integration of β,γ-CH₂-2-MeS-ATP and 2-MeS-AMP) with time were fit to a pseudo first-order exponential decay rate equation with respect to the concentration of 2. The half life determined at pH 1.4/37° C. for 2 was 65 h, as shown in FIG. 1B.

### Example 5

### The Chemical Stability of Compounds 3 and 4, Evaluated by HPLC

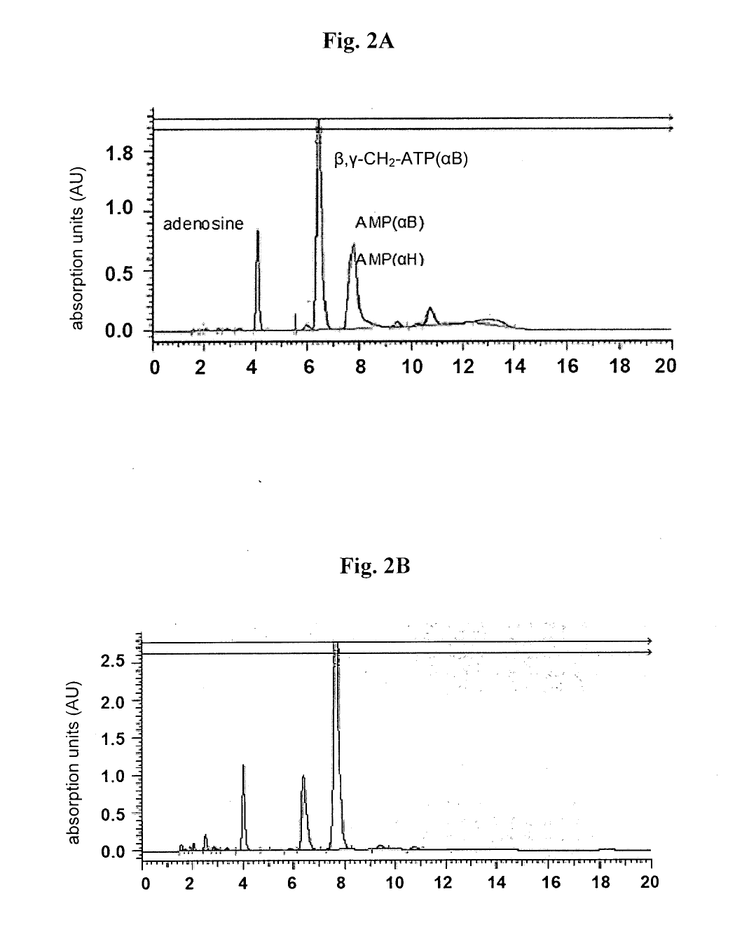

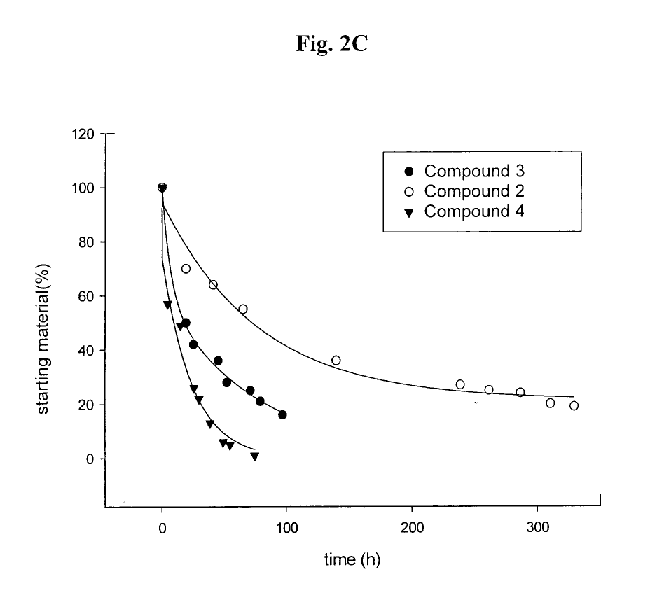

The stability of 3 (isomer B) in the appropriate buffer solution (0.2 M HCl/KCl, pH=1.4) at 37° C. was evaluated by HPLC-electrospray ionization (ESI)MS for 5 days at 7-17 h intervals, and its hydrolysis rate, based on the HPLC integration changes of the 3B peak with time, was fit to a pseudo first-order exponential decay rate equation, as shown in FIGS. 2A-2B. In addition to 3B, degradation products 6, 7 and 8 were identified in the hydrolysis mixture, as depicted in Scheme 3. For instance, after 19 h, 50% of 3B was degraded giving rise to 37% of AMP-α-B and AMP-α-H (6 and 7, respectively. Both appear at the same retention time; however, MS enabled the identification of these compounds) and 13% adenosine (FIG. 2A). The composition change of the hydrolysis mixture with time is depicted in FIG. 2C. The half-life of 3B was 19 h.

The stability of 4 (isomer B) in the appropriate buffer solution (pH=1.4) at 37° C. was evaluated by HPLC to monitor possible dephosphorylation products. Compound 4 (1.6 mg) was dissolved in 0.2 M HCl/KCl buffer (0.4 ml), and the final pH was adjusted to 1.4 by adding 0.2 M HCl (15 μl). The solution was kept in an oil bath at 37° C. and its composition was analyzed by HPLC-MS, using a Gemini analytic column (5u C-18 110A, 150×4.60 mm) and gradient elution with Solvent System I (see Example 1) at 89:11 A:B for 15 min and then 82:18 to 74:26 A:B for 20 min at a flow rate of 1 ml/min. Samples were taken at 12 h intervals for 5 days. The hydrolysis rate of 4 was determined by measuring the change in the integration of the HPLC peaks of the degradation products, 6, 7 and 8, with time. As shown in FIG. 2C, the half-life of compound 4B was 14.5 h.

### Example 6

### The Enzymatic Stability of Compounds 2-4 to Alkaline Phosphatase

Enzyme activity was measured by the release of p-nitrophenol from the nucleotide derivatives using a UV-vis spectrophotometer at 405 nm. Relative activity and resistance of nucleotides to enzymatic hydrolysis were determined at 37° C. Briefly, 32.5 μl of nucleotide derivative (77 μg/ml in 0.1 M Tris-HCl and 0.1 M MgCl₂, pH 7.5) and 6 μl of deionized water were incubated with calf intestine alkaline phosphatase (Fermentas Inc., Glen Burnie, Md., 1 unit/μl, 6.25 μl at 37° C. final pH=9.8). After 30 min, the reaction was stopped by incubation at 80° C. for 30 min. The stability of the nucleotide derivative was evaluated by HPLC to monitor possible dephosphorylation products. The mixture was separated on a Gemini analytic column (5u C-18 110A, 150×4.60 mm) using gradient elution with Solvent System I (see Example 1) at 90:10 to 70:30 A:B for 3A and 3B, and 82:18 to 50:50 for 4A, 4B and 2 over 20 min and at a flow rate of 1 ml/min. The hydrolysis rate was determined by measuring the change in the integration of the respective HPLC peaks with time, and as observed, analogues 2-4 remained completely intact under these conditions.

### Example 7

### The Stability of ATP and Compounds 2-4 in Human Blood Serum

Preparation of the Human Blood Serum: Blood Taken from Healthy Volunteers was obtained from a blood bank (Tel-Hashomer hospital, Israel), stored for 12 h at 4° C. and centrifuged in plastic tubes at 1500 g for 15 min at room temperature. The serum was separated and stored at −80° C.

Evaluation of the Stability of 2-3 in Human Blood Serum, Method A

The assay mixture, containing a 40 mM nucleotide derivative solution in deionized water (4.5 μl), human blood serum (180 μl) and RPMI-1640 (540 μl), was incubated at 37° C. for 1, 4, 8, 16, 24, 48, 72 and 96 h. The samples were then treated with 0.6 M of hydrochloric acid (430 μl), centrifuged for 2 min (13,000 g, 4° C.), neutralized by addition of 4 M of KOH, centrifuged for 2 min (13,000 g, 4° C.) and freeze-dried. The stability of the nucleotide was evaluated by HPLC for monitoring possible dephosphorylation products. The mixture was separated on a Gemini analytic column (5u C-18 110A, 150×4.60 mm) with gradient elution [0.01 M KH₂PO₄ pH=4.5 (A)/acetonitrile (B), 100:0→60:40, A:B, 20 min for 2, 3A and 3B; 100:0→95:5, A:B, 10 min for ATP] and flow rate of 1 ml/min. The hydrolysis rate was determined by measuring the change in the integration of the respective HPLC peaks with time.

Evaluation of the Stability of 4 in Human Blood Serum, Method B

The assay mixture, containing a 40 mM nucleotide derivative solution in deionized water (4.5 μl), human blood serum (180 μl) and RPMI-1640 (540 μl), was incubated at 37° C. for 1, 4, 8, 16, 24, 48, 72, 96, 120 and 144 h. The samples were then heated to 80° C. for 30 min, treated with CM Sephadex (1-2 mg) for 2 h, centrifuged for 6 min (12,000 rpm) and extracted with chloroform (2×500 μl). The aqueous layer was freeze-dried. The stability of the nucleotide was evaluated by HPLC for monitoring possible dephosphorylation products. The mixture was separated on a Gemini analytic column (5u C-18 110A, 150×4.60 mm) with gradient elution [100 mM TEAA, pH 7 (A)/MeOH (B), 79:21, A:B, 15 min for 4A and 4B; 100 mM TEAA, pH 7 (A)/acetonitryl (B), A:B, 10 min, 100:0→90:10 A:B 10 min, 90:10→80:20 A:B 4 min, 80:20 A:B 1 min for ATP] and flow rate of 1 ml/min. The hydrolysis rate was determined by measuring the change in the integration of the respective HPLC peaks with time.

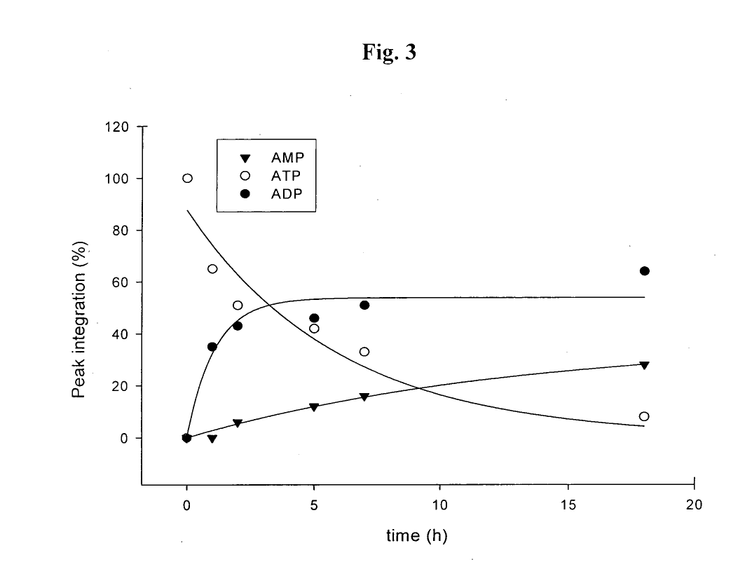

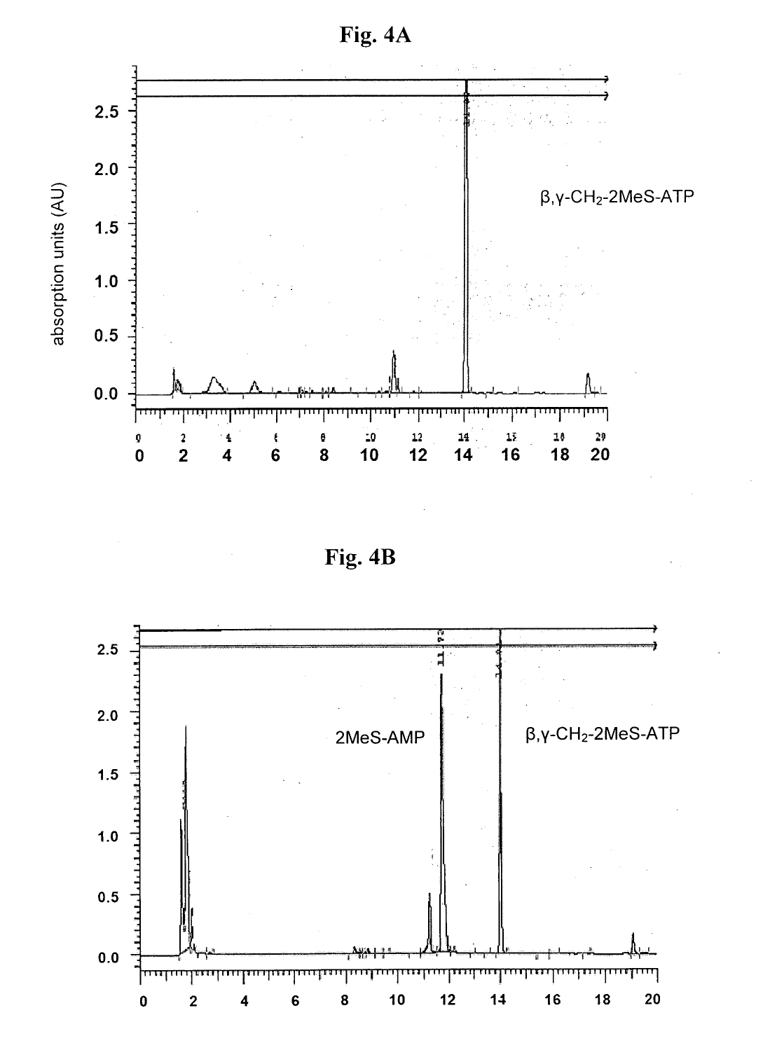

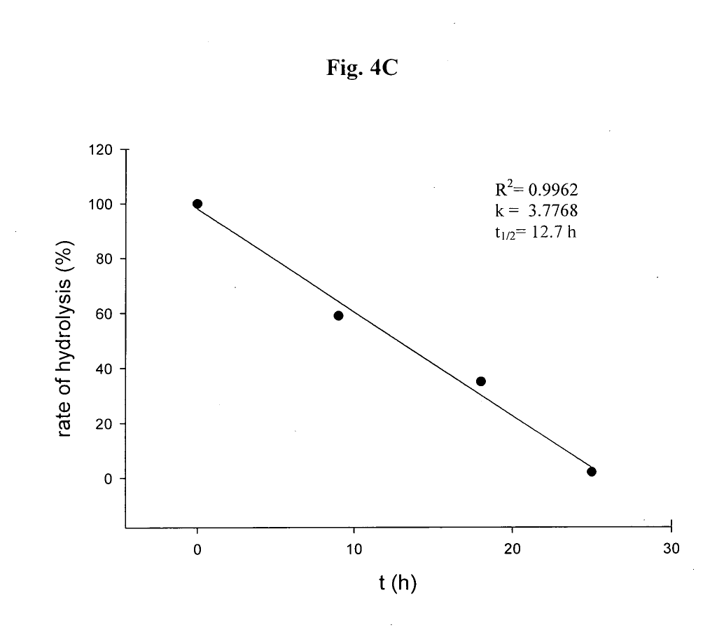

As shown in FIG. 3, ATP was hydrolyzed to ADP and AMP with half-life of 3.6 h (Method A), while under the same conditions, compounds 2, 3A and 3B were mostly hydrolyzed to the corresponding nucleoside-monophosphate (boranophosphate) with half-lives of 12.7, 14.1 and 47.1 h, respectively (data regarding the hydrolysis rate of compound 2 is shown in FIGS. 4A-4C). Compound 4B was hydrolyzed with half-life of 71.9 h (Method B), while under the same conditions, ATP was hydrolyzed with half-life of 7.7 h. These values represent a 3.5-20-fold substitution-dependent enhancement of the metabolic stability of ATP.

### Example 8

### 2-MeS-adenosine-5′-dihalogenomethylene-diphosphate, 17-18, synthesis

In order to prepare 2-MeS-adenosine-5′-dichloromethylene-diphosphate and 2-MeS-adenosine-5′-difluoromethylene-diphosphate, 17 and 18 respectively, 5′-O-tosyl-2′,3′-O-acetonide-2MeS-adenosine, 16, was first prepared as depicted in Scheme 4 and described hereinbelow:

2′, 3′ acetonide-2-MeS-adenosine, 15 (Nahum et al., 2002), (97 mg, 0.27 mmol) was dissolved in dry dichloromethane (1 ml) in a flame-dried two-neck flask under N₂. A solution of DMAP (134 mg, 1.09 mmol, 4 eq) in dry dichloromethane (2 ml) and a solution of TsCl (156 mg, 0.82 mmol, 3 eq) in dry dichloromethane (0.5 ml) were added and the solution obtained was stirred at room temperature over 12 h under N₂. The reaction was diluted with dichloromethane (50 ml) and extracted with a saturated solution of NaHCO₃ (3×30 ml). The organic layer was removed, dried with Na₂SO₄, filtered and evaporated under reduced pressure to obtain a white solid, which was separated on MPLC system using a silica gel column [25+M column, using the following gradient scheme: CHCl₃ (A): EtOH (B), 0:0, 3 CV, A:B, CHCl₃ (A): EtOH (B), 0:0→0:10, 5 CV, A:B, CHCl₃ (A): EtOH (B), 10:0, 4 CV, at a flow rate of 25 ml/min]. Compound 16 was obtained in 52% yield. ¹H NMR (CHCl₃, 300 MHz): δ 7.68 (s, H-8, 1H), 7.72 (d, J=8.4 Hz, 2H) 7.27 (d, signal is hidden by the d-chloroform) 6.05 (d, J=7.2 Hz, H-1′, 1H), 6.35 (t, J=5.4 Hz, H2′), 5.00 (q, J=5.70, H-3′, 1H), 4.40 (m, H-4′, 1H), 4.25 (m, H-5′, H-5″ 2H), 2.59 (s, 3H), 2.43 (s, 3H), 1.61 (s, 3H), 1.38 (s, 3H). MS-ES+ m/z: 508 (M⁺). TLC (EtOH:CHCl₃ 5:95), R_{f}=0.79.

Compound 16 (42 mg, 0.08 mmol) was then dissolved in dry DMF (0.2 ml) in a flame-dried two-neck flask under N₂. A solution of tris(tetrabutylammonium)dihalogenemethylenediphosphonate (0.16 mmol, 2 eq) in dry DMF (0.3 ml) was added and the solution was stirred at room temperature for 72 h. TFA neat (2 ml) was added and the reaction was stirred at room temperature for 10 min under N₂ bubbling. The solvent was removed under reduced pressure to obtain a yellow solid, which was separated on an activated Sephadex DEAE-A25 column (0-0.3 M NH₄HCO₃, total volume of 1.4 l). The relevant fractions containing either compound 17 or 18 were collected and freeze-dried, and excess NH₄HCO₃ was removed by repeated freeze-drying cycles with deionized water.

### Example 9

### 2-SMe-adenosine-5′-O-(Pα-borano) diphosphate, 19, synthesis, separation and characterization

As depicted in Scheme 5, 2′,3′-methoxy methylidene nucleoside (490.4 mg, 1.38 mmol) was dissolved in DMF (3 ml)/pyridine (0.6 ml, 5 eq) in a flame dried septum-sealed flask under Argon. A freshly prepared solution of salicylphosphochloridite (307 mg, 1.1 eq) in dioxane (1 ml) was then transferred to the flask through a syringe. After stirring for 10 min at room temperature, a freshly prepared 1 M solution of bis(tri-n-butylammonium)pyrophosphate (2.1 ml, 1.5 eq) in DMF and tri-n-butylamine (1.3 ml, 4 eq) were simultaneously injected through the septum. A 2 M solution of BH₃:SMe₂ complex in THF (7 ml, 10 eq) was added to the flask and the mixture was stirred for 15 min at room temperature. Ethylenediamine (0.5 ml, 5 eq) was then injected to the flask through a syringe. After stirring for 60 min, deionized water (4 ml) was added to the flask. After 10 min, the reaction mixture was evaporated. The residue was diluted by deionized water and extracted with ethyl ether. The aqueous layer was then freeze-dried and the resulting residue was applied on an activated Sephadex DEAE-A25 column (0-0.4 M NH₄HCO₃, total volume 900 ml). The relevant fractions were collected and freeze-dried; excess NH₄HCO₃ was removed by repeated freeze-drying with deionized water to yield compound 19 as the ammonium salt. The methoxymethylidene protecting group was removed by acidic hydrolysis (10% HCl solution was added till pH 2.3 was obtained). After 3 h at room temperature, the pH was rapidly raised to 9 by the addition of NH₄OH solution (pH 11) and the solution was kept at room temperature for 40 min. Compound 19 was obtained at a 46% yield after LC separation. Final purification and separation of diastereoisomers were achieved by HPLC, isocratic elution with TEAA:acetonitrile 88:12.

2MeS-adenosine-5′-O-(Pα-borano) diphosphate, 19A, characterization

Retention time: 8.073 min. ³¹P NMR (D₂O, 81 MHz, pH 7): δ 82.5 (m, Pα-BH₃, 1P), −9.5 (d, Pβ, 1P) ppm. ¹H NMR (D₂O, 200 MHz): δ 8.55 (s, H-8, 1H), 6.25 (d, H-1′, 1H), 4.6 (dd, H-3′, 1H), 4.35 (q, H-4′, 1H), 2.7 (s, CH₃—S, 3H), 0.3 (m, BH₃, 3H) ppm. Purity data obtained on an analytical column: Retention time: 4.113 min (98% purity) using Solvent System III (100 mM TEAA, pH 7 (A)/acetonitrile (B), 88:12 A:B, flow rate of 1 ml/min). Retention time: 3.158 min (98.5% purity) using solvent system IV (0.01 M KH₂PO₄, pH 4.5 (A)/acetonitrile (B), 90:10 A:B, flow rate of 1 ml/min).

2MeS-adenosine-5′-O-(Pα-borano) diphosphate, 19B, characterization

Retention time: 9.127 min. ³¹P NMR (D₂O, 81 MHz, pH 7): δ 82.5 (m, Pα-BH₃, 1P), −9.0 (d, Pβ, 1P), −22.5 (dd, Pβ, 1P) ppm. ¹H NMR (D₂O, 200 MHz): δ 8.50 (s, H-8, 1H), 6.20 (d, H-1′, 1H), 4.5 (dd, H-3′, 1H), 4.30 (q, H-4′, 1H), 2.6 (s, CH₃—S, 3H), 0.3 (m, BH₃, 3H) ppm. Purity data obtained on an analytical column: Retention time: 4.720 min (95% purity) using Solvent System III (see hereinabove). Retention time: 3.764 min (94% purity) using Solvent System IV (see hereinabove).

### Example 10

### The stability of 2MeS-adenosine-5′-O-(Pα-borano) diphosphate, 19, to alkaline phosphatase and in human blood serum

The stability of compound 19 (isomer A) to alkaline phosphatase was measured as described in Example 6 and its t_{1/2} was found to be about 6 h vs. about 4 h for ADP.

In addition, the stability of this compound was measured in human blood serum and as found, this compound was hydrolyzed with half-life of >24 h vs. about 2 h for ADP. In particular, the percentage of the compound hydrolyzed after 24 h was about 25-40% only.

### Example 11

### Compounds 2, 4B, 17, 18, 19A, 21A and 21B as potential agonists of P2Y_{1/6} receptors

The activities of compounds 2, 4B, 17, 18, 19A, 21 (isomers A and B) were examined at the G protein-coupled P2YRs, P2Y₁, P2Y₂, P2Y₄ and P2Y₆ expressed in human 1321N1 astrocytoma cells, based on intracellular calcium measurements, as described in Experimental above. In particular, the activity of compound 2, 4B and 19 was examined at P2Y₁R, P2Y₂R, P2Y₄R and P2Y₆R, and the activity of compounds 17, 18 and 21 was examined at P2Y₁R only. The experiments were conducted by Prof. Gary A. Weisman from Columbia-Missouri University, Columbia, Mo., USA.

As shown in Table 1 hereinbelow, compound 19A was found to be the most potent and selective agonist of the P2Y₁R with EC₅₀'s of 0.038 μM as compared to 0.004 μM for 2-MeS-ADP. Compounds 2 and 4B were agonists of the P2Y₁R with EC₅₀'s of 0.08 and 17.2 μM, respectively, and had a slight agonistic effect at 100 μM on P2Y₆R. Compounds 17, 18, 21A and 21B were agonists of the P2Y₁R with EC₅₀'s of 3.1, 0.98, 0.57 and 1.2 μM, respectively.

TABLE 1
Activity of compounds 2, 4B, 17, 18, 20A and 20B at P2Y_{1/2/4/6}R

| | Compound-induced activation of [Ca²⁺] | | | |
|---|---|---|---|---|
| | EC₅₀ (μM) values. | | | |
| Compound | P2Y₁ | P2Y₂ | P2Y₄ | P2Y₆ |
| 2 | 0.08 ± 0.03 | nr | nr | sr |
| 4B | 17.2 ± 5.3 | nr | nr | sr |
| 17 | 3.1 | | | |
| 18 | 0.98 | | | |
| 19A | 0.038 | nr | nr | nr |
| 21A | 0.57 | | | |
| 21B | 1.2 | | | |
| 2-MeS-ADP | 0.004 ± 0.002 | | | |
| UTP | | 0.64 ± 0.25 | 0.48 ± 0.31 | |
| UDP | | | | 0.20 ± 0.06 |

* sr = slight response at 100 μM;
nr = no response

### Example 12

### In Vivo Study of the Efficacy of the Compounds of the Present Invention as Insulin Secretagogues

Paradigm

The objective of this experiment is to study in vivo the efficacy of the compounds of the present invention as insulin secretion enhancing molecules, following a single oral gavage (per os) administration of glucose to cannulated Wister rats, by measuring the blood glucose and insulin levels following administration of the tested compound to the cannulated rats intra venues through the cannule.

A total of about 40 healthy 10-13 week old Wister rats are used. Animals are acclimatized for at least 4 days prior to commencement of treatment and are fed with non-medicated commercial sterile rodent food ad libitum. Drinking tap water are available ad libitum.

About 48 hours prior treatment, rats are weighted and a population uniform in weight (about 90% of the animals) is taken for cannulation. In particular, animals are anesthetized by 2.5% isofluran 97.5% dry air inhalation, and a P52 cannula is surgically inserted and fixed in the jugular vein and flushed with 0.3-0.5 ml 5% heparinized saline after cannulation (and thereafter, immediately after each blood collection). In case a technical problem such as fixation or clotting arises with a rat's indwelling cannula, another rat is cannulated and replace the previously allocated rat in the study.

On the day of treatment, the cannule of each one of the rats is checked, the rats are weighed, and the glucose level of each one of the rats is checked via the tail vain. A population uniform in glucose levels and weight is divided into three groups, wherein the first group is treated with the tested compound; the second group is a negative control group treated with saline; and the third group is a positive control group treated with glibenclamide, also known as glyburide. The latter is an anti-diabetic drug classified as sulfonylurea, used in the treatment of type II diabetes, which currently is one of only two oral anti-diabetics in the WHO Model List of Essential Medicines. Glibenclamide works by inhibiting ATP-sensitive potassium channels in pancreatic beta cells, causing cell membrane depolarization and opening of voltage-dependent calcium channels, thus triggering an increase in intracellular calcium into the beta cell that stimulates insulin release.

All rats participating in the experiment are administered (per os) with a glucose challenge of 2 g/kg body, wherein the total volume of glucose administration per rat is 3 ml/kg body weight from a solution of 0.67 g/ml. Ten minutes following the glucose administration, the rats of the first group are administered with the tested compound; and the rats of the negative control group are administered with saline. In both cases, administration is performed IV via the cannule. The administered dose-level of the tested compound is 2.5 mg/kg body weight and the volume for administration is 1 ml/kg body weight; and the volume of the saline administered is 1 ml/kg body weight. The rats of the positive control group are administered (per os) with glibenclamide, 30 minutes prior to glucose administration. The administered dose-level of glibenclamide is 1 mg/kg body weight and the volume for administration is 5 ml/kg body weight from a solution of 0.2 mg/ml. Post dosing, the rats are placed back in the cage pending blood samplings.

Glucose and insulin levels are measured 30 minutes prior to glucose administration (and prior to glibenclamide administration in the case of the positive control group); immediately before and 5 minutes after glucose administration; 15 minutes after glucose administration, namely, 5 minutes after administration of the tested compound or saline (in the cases of the first and the negative control groups, respectively); and then 30, 45, 60 120 and 150 minutes after glucose administration.

For glucose levels measurement, blood samples are withdrawn from each rat via the tail vein and are immediately tested with a glucometer. For insulin levels measurement, blood samples are withdrawn from each rat via the jugular vein cannula. The volume of blood collected from each treated rat is 150 μl. Blood samples withdrawn for insulin levels are collected into 0.8 ml tubes with Z serum/Gel. Blood is left to clot at room temperature for at least 30 minutes, and post clotting, it is centrifuged (3000×g, 15 min) at approximately 4° C. Serum is harvested and equally divided (at least 25 μl per aliquot) between two 0.2 ml flat cap PCR tubs, and is then stored frozen at −20° C. until analyzed. Clinical observations are performed post each individual animal's dosing and within bleeding period. Blood analysis of glucose levels in whole blood is done on site, during bleeding, using a blood glucose monitoring system on test strips suitable for this system. Blood analysis of insulin levels in serum is done using Rat/Mouse insulin kit.

The in-life phase of this experiment is completed 150 minutes post glucose administration, following the final blood sample collection and serum harvesting.

It is expected that rats treated with the tested compound will have significantly lower levels of glucose in the blood samples taken, starting from 15 minutes following glucose administration, i.e., 5 minutes after administration of the tested compound. The levels of glucose expected are, in fact, very similar to the glucose levels measured following starvation.

It is further expected to see significant increase in the insulin levels of the rats treated with the tested compound, already about 15 minutes following glucose administration, i.e., about 15 minutes prior to the increase in insulin level normally measured in healthy individuals following glucose administration. The increased level of insulin is expected to be maintained during 30-45 minutes and then to decrease at a certain rate, depending on the stability of the tested compound in the blood.

Results

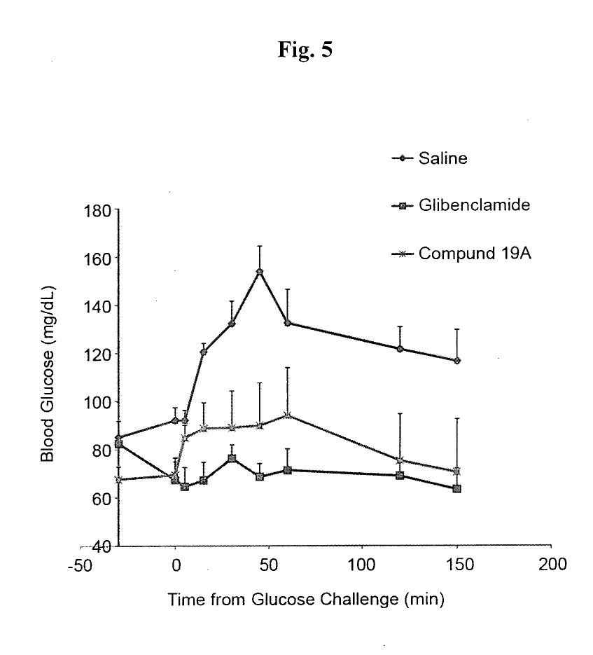

In a preliminary study, 2MeS-adenosine-5′-O-(Pα-borano) diphosphate, 19 (2.5 mg/kg), was intravenously administered to starved Wistar rats (n=5) as described hereinabove, while saline was administered to the rats of the negative control group and glibenclamide (0.25 mg/kg) was given 30 minutes prior to glucose administration to the rats of the positive control group. As shown in FIG. 5, compound 19 reduced the glucose levels measured relative to the that measured in the saline treated rats, similarly to glibenclamide.

### APPENDIX A

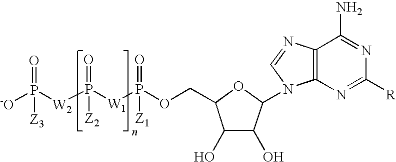

| Compound | R | Z₁ | Z₂, Z₃ | W₁ | W₂ | n |
|---|---|---|---|---|---|---|
| 1 | SMe | BH³⁻ | O⁻ | O | O | 1 |
| 2 | SMe | O⁻ | O⁻ | O | CH₂ | 1 |
| 3 | H | BH³⁻ | O⁻ | O | CH₂ | 1 |
| 4 | SMe | BH³⁻ | O⁻ | O | CH₂ | 1 |
| 20 | H | BH³⁻ | O⁻ | O | CCl₂ | 1 |
| 21 | SMe | BH³⁻ | O⁻ | O | CCl₂ | 1 |
| 22 | SMe | BH³⁻ | O⁻ | O | CF₂ | 1 |
| 17 | SMe | O⁻ | —, O⁻ | — | CCl₂ | 0 |
| 18 | SMe | O⁻ | —, O⁻ | — | CF₂ | 0 |
| 19 | SMe | BH³⁻ | —, O⁻ | — | O | 0 |

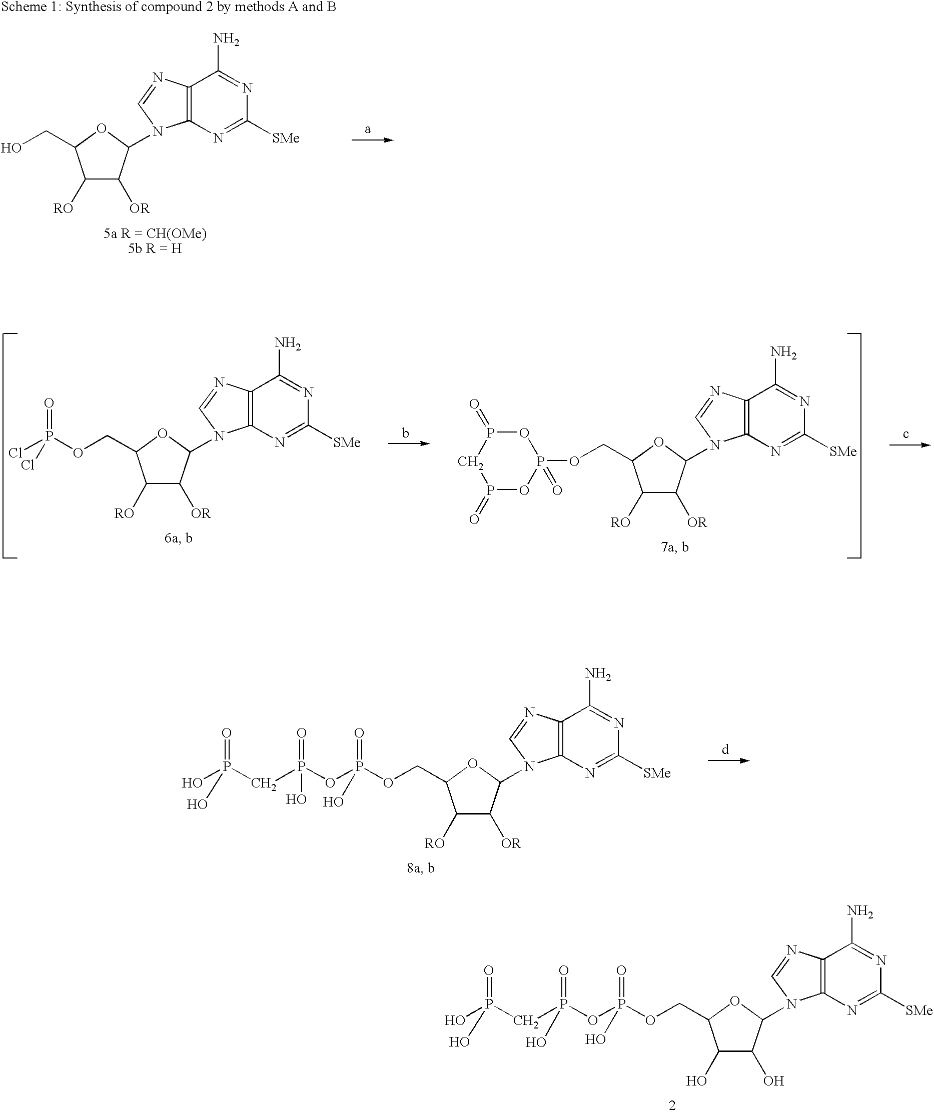

### Reaction Conditions:

Method A: starting from 5a a) trimethylphosphate, POCl₃, Proton Sponge™, 0° C., 3 h; b) 0.5 M bis(tributylammonium)methylenediphosphonate in dry DMF, Bu₃N, 0° C., 1.6 min; c) 0.5 M TEAB, pH=7, rt, 0.5 h; and d) 1) 18% HCl, pH 2.3, rt, 3 h; and 2) 24% NH₄OH, pH 9, rt, 45 min.
Method B: starting from 5b a) trimethylphosphate, POCl₃, Proton Sponge™, 0° C., 2 h; b) 1 M bis(tributylammonium)methylenediphosphonate in dry DMF, Bu₃N, 0° C., 25 min; and c) 0.5 M TEAB, pH 7, rt, 0.5 h.

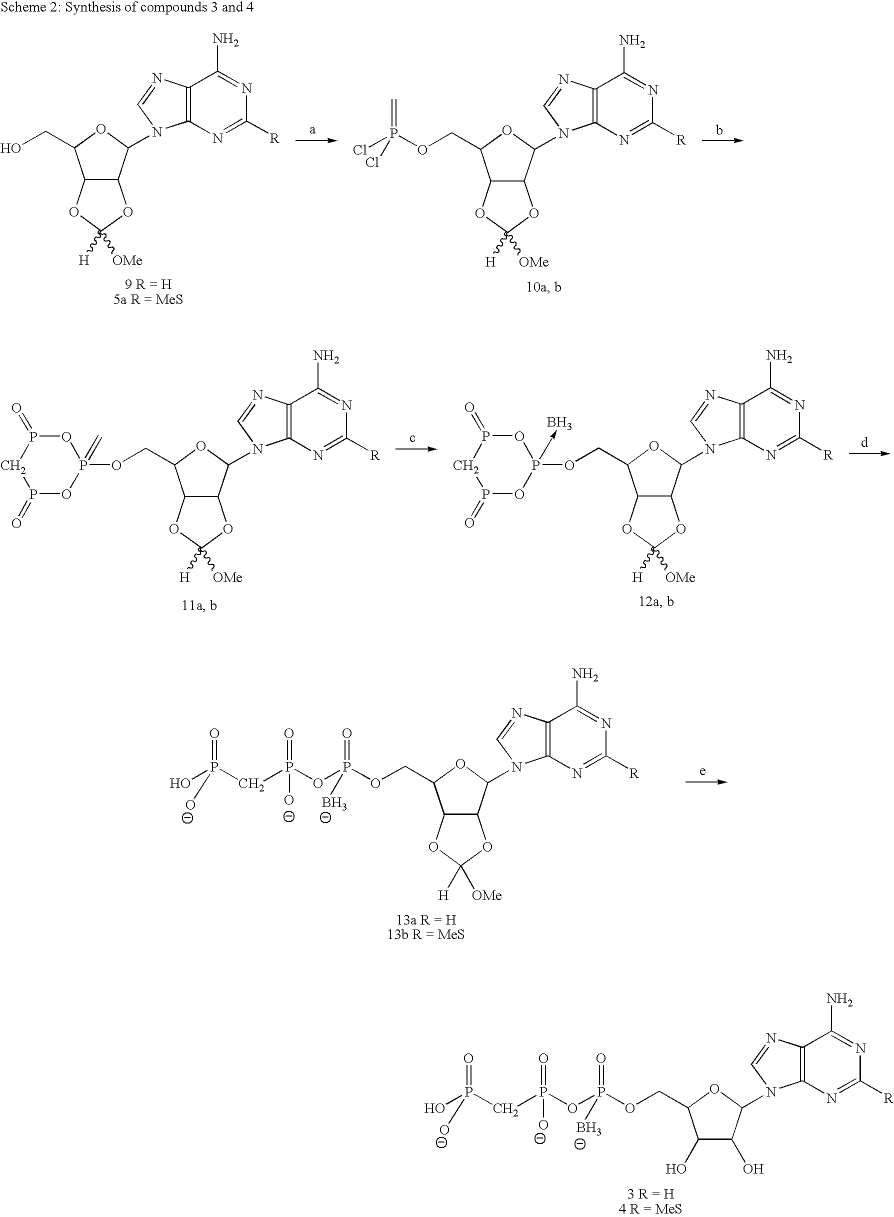

Reaction conditions: a) trimethylphosphate, PCl₃, Proton Sponge™, 0° C., 30 min; b) 1 M bis(tributylammonium)methylenediphosphonate in dry DMF, Bu₃N, 0° C., 11 min; c) 2 M BH₃.SMe in THF, 0° C., 5 min then rt, 30 min; d) 1 M TEAB, pH 7, rt, 0.5 h; and e) 1) 18% HCl, pH 2.3, rt, 3 h; and 2) 24% NH₄OH, pH 9, rt, 45 min.

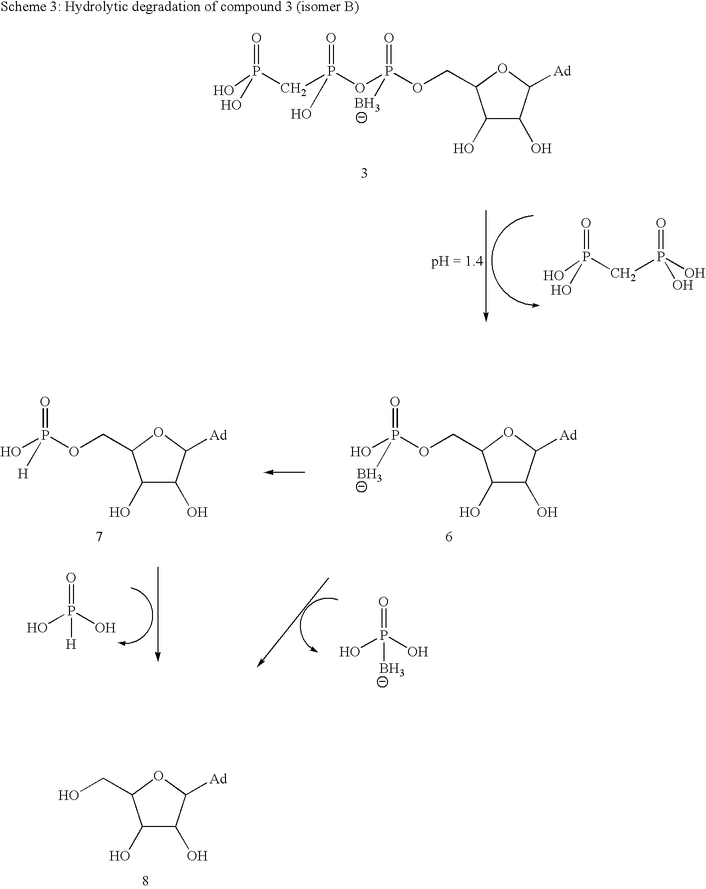

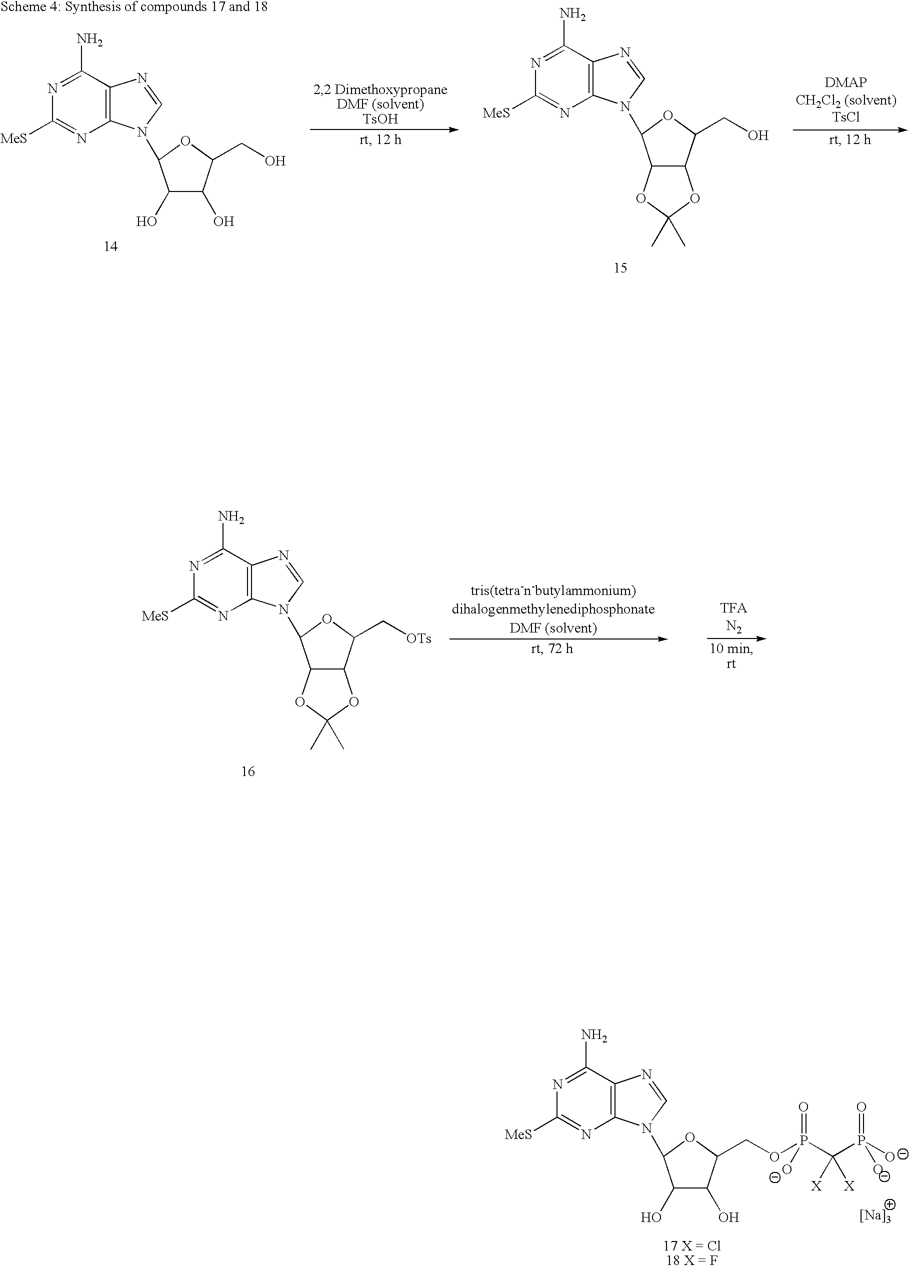

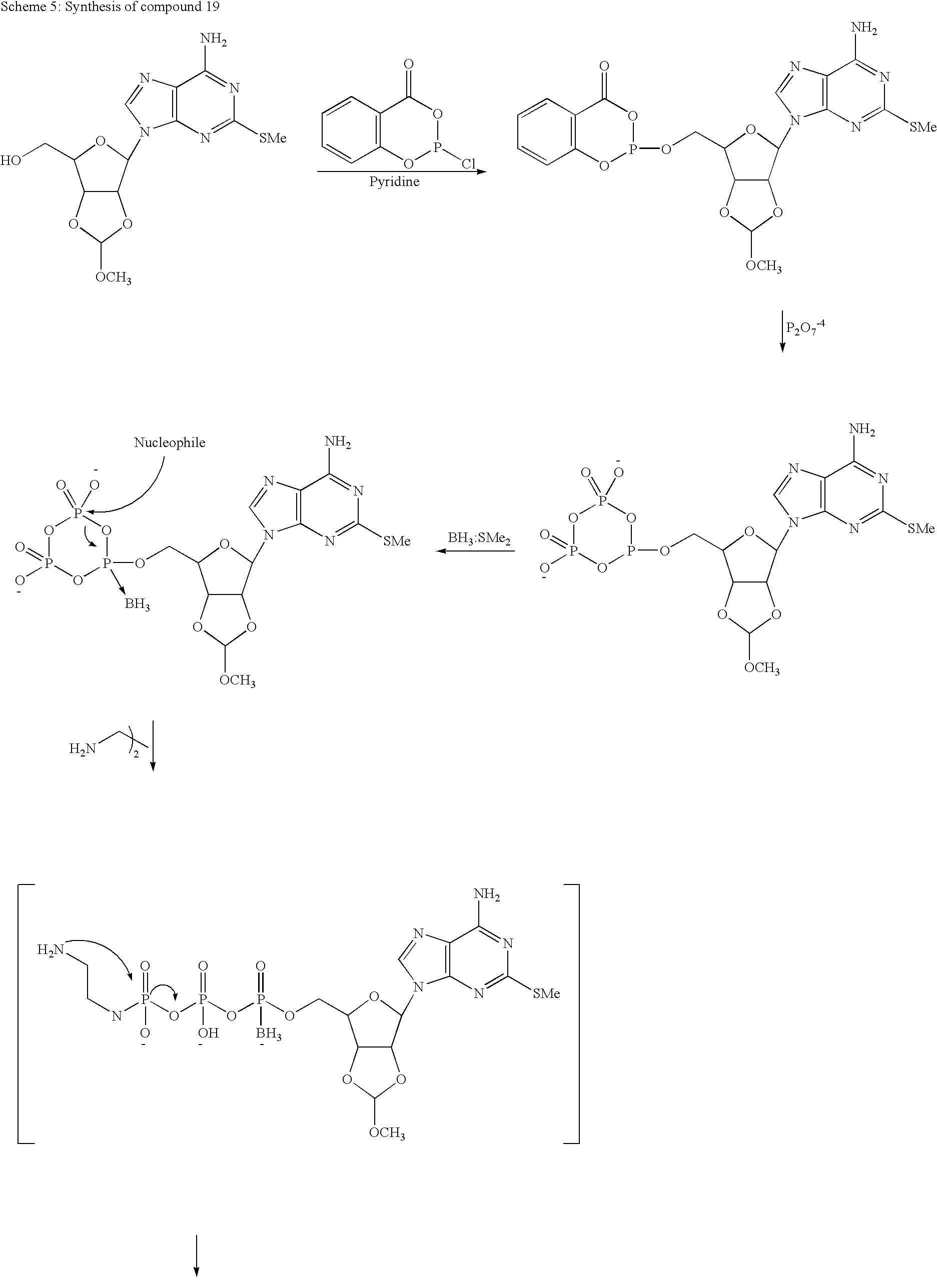

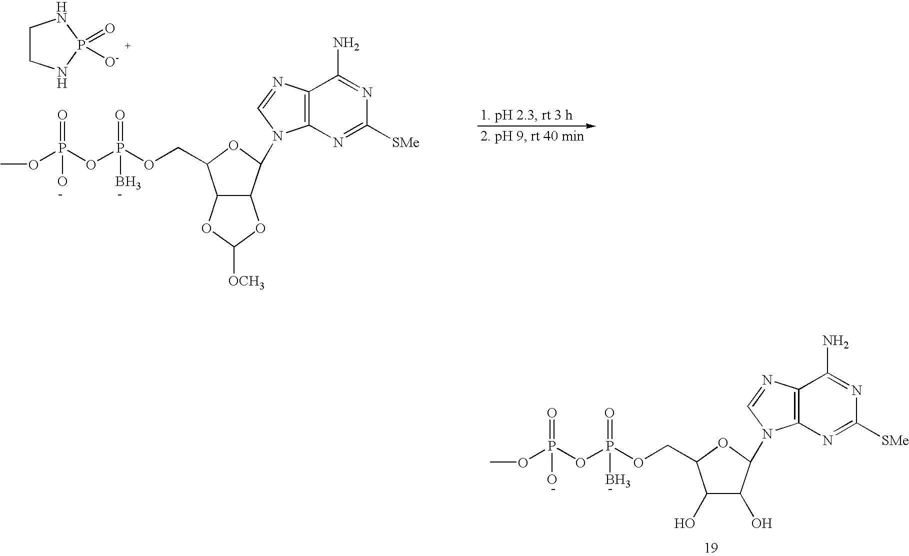

### REFERENCES

Abbracchio M. P., Burnstock G., Boeynaems J. M., Barnard E. A., Boyer J. L., Kennedy C., Knight G. E., Fumagalli M., Gachet C., Jacobson K. A., Weisman G. A., Pharmacological Reviews, 2006, 58, 281-341
Adams D. J., Drug Dev. Res., 1994, 31, 242
Angiolillo D. J., Bernardo E., Ramirez C., Costa M. A., Sabate M., Jimenez-Quevedo P., Hernandez R., Moreno R., Escaned J., Alfonso F., Banuelos C., Bass T. A., Macaya C., Fernandez-Ortiz A., Journal of the American College of Cardiology, 2006a, 48, 298-304
Angiolillo D. J., Fernandez-Ortiz A., Bernardo E., Ramirez C., Sabate M., Jimenez-Quevedo P., Hernandez R., Moreno R., Escaned J., Alfonso F., Banuelos C., Costa M. A., Bass T. A., Macaya C., Diabetes, 2006b, 55, 780-784
Arzumanov A. A., Semizarov D. G., Victorova L. S., Dyatkina N. B., Krayevsky A. A., Journal of Biological Chemistry, 1996, 271, 24389-24394
Blackburn G. M., Taylor G. E., Thatcher G. R. J., Prescott M., McLennan A. G., Nucleic Acids Research, 1987, 15, 6991-7004
Buckley M. F., Loveland K. A., McKinstry W. J., Garson O. M., Goding J. W., J Biol Chem, 1990, 265, 17506-17511
Burnstock G., Kennedy C., Gen. Pharmacol., 1985, 16, 433-440
Burnstock G., Fischer B., Maillard M., Ziganshin A., Ralevic V., Knight G., Brizzolara A., von Isakovics A., Boyer J. L., Harden T. K., Jacobson K., Drug Dev. Res., 1994, 31, 206-219
Bystrom C. E., Pettigrew D. W., Remington S. J., Branchaud B. P., Bioorganic & Medicinal Chemistry Letters, 1997, 7, 2613-2616
Chen B. C., Lin W. W., Biochemical and Biophysical Research Communications, 1997, 233, 442-446
Chow G., Ziegelstein R. C., Am J Cardiovasc Drugs, 2007, 7, 169-171
Cusack N. J., Hourani S. M., Loizou G. D., Welford L. A., Br J Pharmacol, 1987, 90, 791-795
Dyatkina N., Shirokova E., Theil F., Roberts S. M., Krayevsky A., Bioorganic & Medicinal Chemistry Letters, 1996, 6, 2639-2642
El-Tayeb A., Griessmeier K. J., Mueller C. E., Bioorganic & Medicinal Chemistry Letters, 2005, 15, 5450-5452
Evans R. J., Kennedy C., Drug Dev. Res., 1994, 31, 285
Farret A., Filhol R., Linck N., Manteghetti M., Vignon J., Gross R., Petit P., Pharm Res, 2006, 23, 2665-2671
Fischer B., Boyer J. L., Hoyle C. H., Ziganshin A. U., Brizzolara A. L., Knight G. E., Zimmet J., Burnstock G., Harden T. K., Jacobson K. A., J Med Chem, 1993, 36, 3937-3946
Fischer B., Exp Opin Therap Patents, 1999, 9, 385-399
Garrad R. C., Otero M. A., Erb L., Theiss P. M., Clarke L. L., Gonzalez F. A., Turner J. T., Weisman G. A., J. Biol. Chem., 1998, 273, 29437-29444
Grynkiewicz G., Poeinie M., Tsien R. Y., J. Biol. Chem., 1985, 260, 3440-3450
Guile S. D., Ince F., Ingall A. H., Kindon N. D., Meghani P., Mortimore M. P., Prog Med Chem, 2001, 38, 115-187
He K., Hasan A., Shaw B. R., Nucleic Acids Symposium Series, 1997, 36, 159
Jacobson K. A., Jarvis M. F., Williams M., J Med Chem, 2002, 45, 4057-4093
Janssens R., Communi D., Pirotton S., Samson M., Parmentier M., Boeynaems J. M., Biochemical and Biophysical Research Communications, 1996, 221, 588-593
Joseph S. M., Buchakjian M. R., Dubyak G. R., Journal of Biological Chemistry, 2003, 278, 23331-23342
Joseph S. M., Pifer M. A., Przybylski R. J., Dubyak G. R., British Journal of Pharmacology, 2004, 142, 1002-1014
Kaczmarek E., Koziak K., Sevigny J., Siegel J. B., Anrather J., Beaudoin A. R., Bach F. H., Robson S. C., Journal of Biological Chemistry, 1996, 271, 33116-33122
Kikuta Y., Ohiwa E., Okada K., Watanabe A., Haruki S., Acta Anaesthesiologica Scandinavica, 1999, 43, 82-86
Kowalska J., Lewdorowicz M., Darzynkiewicz E., Jemielity L., Tetrahedron Letters, 2007, 48, 5475-5479
Labataille P., Pelicano H., Maury G., Imbach J. L., Gosselin G., Bioorganic & Medicinal Chemistry Letters, 1995, 5, 2315-2320
Laxman S., Beavo J. A., Molecular Interventions, 2007, 7, 203-215
Lazarowski E. R., Homolya L., Boucher R. C., Harden T. K., Journal of Biological Chemistry, 1997, 272, 20402-20407
Lazarowski E. R., Boucher R. C., Harden T. K., Journal of Biological Chemistry, 2000, 275, 31061-31068
Lin J., Porter K. W., Shaw B. R., Nucleosides, Nucleotides & Nucleic Acids, 2001, 20, 1019-1023
Macfarlane D. E., Methods Enzymol., 1992, 215, 137-142
Major D. T., Nahum V., Wang Y., Reiser G., Fischer B., J. Med. Chem., 2004, 47, 4405-4416
Maminishkis A., Jalickee S., Blaug S. A., Rymer J., Yerxa B. R., Peterson W. M., Sheldon S. M., Invest Opthalmol Vis Sci, 2002, 43, 3555-3566
McGuigan C., Sheeka H. M., Mahmood N., Hay A., Bioorganic & Medicinal Chemistry Letters, 1993, 3, 1203-1206
McGuigan C., Cahard D., Sheeka H. M., De Clercq E., Balzarini J., Bioorganic & Medicinal Chemistry Letters, 1996a, 6, 1183-1186
McGuigan C., Tsang H. W., Mahmood N., Hay A. J., Antiviral Chemistry & Chemotherapy, 1996b, 7, 330-337
Misiura K., Szymanowicz D., Stec W. J., Organic Letters, 2005, 7, 2217-2220
Mohamady S., Jakeman D. L., Journal of Organic Chemistry, 2005, 70, 10588-10591
Mundasad M. V., Novack G. D., Allgood V. E., Evans R. M., Gorden J. C., Yerxa B. R., Journal of Ocular Pharmacology and Therapeutics, 2001, 17, 173-179
Myers T. C., Nakamura K., Flesher J. W., Journal of the American Chemical Society, 1963, 85, 3292-3295
Nahum V., Zuendorf G., Levesque S. A., Beaudoin A. R., Reiser G., Fischer B., Journal of Medicinal Chemistry, 2002, 45, 5384-5396
Nahum V., Tulapurkar M., Levesque S. A., Sevigny J., Reiser G., Fischer B., J Med Chem, 2006, 49, 1980-1990
Padyukova N. S., Dixon H. B. F., Efimtseva E. V., Ermolinsky B. S., Mikhailov S. N., Karpeisky M. Y., Nucleosides & Nucleotides, 1999, 18, 1013-1014
Picher M., Sevigny J., D'Orleans-Juste P., Beaudoin A. R., Biochemical Pharmacology, 1996, 51, 1453-1460
Romaniuk P. J., Eckstein F., Journal of Biological Chemistry, 1981, 256, 732:2-7328
Sak K., Raidaru G., Webb T. E., Jary J., Archives of Biochemistry and Biophysics, 2000, 381, 171-172
Shaver S. R., Rideout J. L., Pendergast W., Douglass J. G., Brown E. G., Boyer J. L., Patel R. I., Redick C. C., Jones A. C., Picher M., Yerxa B. R., Purinergic Signalling, 2005, 1, 183-191
Shirokova E. A., Dyatkina N. B., Collection of Czechoslovak Chemical Communications, 1996, 61, S158-S160
Spelta V., Mekhalfia A., Rejman D., Thompson M., Blackburn G. M., North R. A., British Journal of Pharmacology, 2003, 140, 1027-1034
Stingelin J., Bolen D. W., Kaiser E. T., Journal of Biological Chemistry, 1980, 255, 2022-2025
Tulapurkar M. E., Laubinger W., Nahum V., Fischer B., Reiser G., Br. J. Pharmacol., 2004, 142
Vollmayer P., Clair T., Goding J. W., Sano K., Servos J., Zimmermann H., Eur J Biochem, 2003, 270, 2971-2978
Wang G., Boyle N., Chen F., Rajappan V., Fagan P., Brooks J. L., Hurd T., Leeds J. M., Rajwanshi V. K., Jin Y., Prhavc M., Bruice T. W., Cook P. D., Journal of Medicinal Chemistry, 2004, 47, 6902-6913
Williams M., Jarvis M. F., Biochem Pharmacol, 2000, 59, 1173-1185
Yanachkov I., Pan J. Y., Wessling-Resnick M., Wright G. E., Molecular Pharmacology, 1997, 51, 47-51
Yegutkin G. G., Burnstock G., Biochimica et Biophysica Acta/Biomembranes, 2000, 1466, 234-244
Yegutkin G. G., Henttinen T., Jalkanen S., FASEB Journal, 2001, 15, 251-260
Yegutkin G. G., Henttinen T., Samburski S. S., Spychala J., Jalkanen S., Biochemical Journal, 2002, 367, 121-128
Yerxa B. R., Sabater J. R., Davis C. W., Stutts M. J., Lang-Furr M., Picher M., Jones A. C., Cowlen M., Dougherty R., Boyer J., Abraham W. M., Boucher R. C., Journal of Pharmacology and Experimental Therapeutics, 2002, 302, 871-880
Zimmermann H., Naunyn-Schmiedeberg's Archives of Pharmacology, 2000, 362, 299-309

1. A compound of the general formula I: 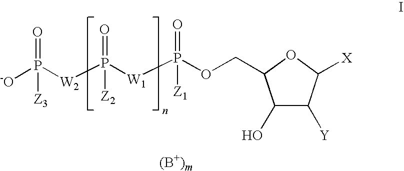
wherein
X is an adenine residue of the formula Ia, linked through the 9-position: 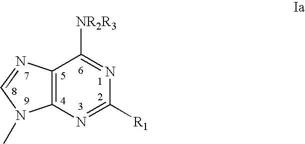
wherein
R₁ is H, halogen, O-hydrocarbyl, S-hydrocarbyl, NR₄R₅, heteroaryl, unsubstituted hydrocarbyl or hydrocarbyl substituted by halogen, CN, SCN, NO₂, OR₄, SR₄, NR₄R₅ or heteroaryl, wherein R₄ and R₅ each independently is H or hydrocarbyl or R₄ and R₅ together with the nitrogen atom to which they are attached form a 5- or 6-membered saturated or unsaturated heterocyclic ring optionally containing 1-2 further heteroatoms selected from oxygen, nitrogen or sulfur, the additional nitrogen being unsubstituted or substituted by alkyl substituted by halogen, hydroxyl or phenyl; and
R₂ and R₃ each independently is H or hydrocarbyl;
or X is an uracil residue of the formula Ib, linked through the 1-position: 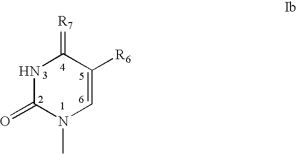
wherein
R₆ is H, halogen, O-hydrocarbyl, S-hydrocarbyl, NR₈R₉, heteroaryl, unsubstituted hydrocarbyl or hydrocarbyl substituted by halogen, CN, SCN, NO₂, OR₈, SR₈, NR₈R₉ or heteroaryl, wherein R₈ and R₉ each independently is H or hydrocarbyl or R₈ and R₉ together with the nitrogen atom to which they are attached form a 5- or 6-membered saturated or unsaturated heterocyclic ring optionally containing 1-2 further heteroatoms selected from oxygen, nitrogen or sulfur, the additional nitrogen being unsubstituted or substituted by alkyl substituted by halogen, hydroxyl or phenyl; and
R₇ is O or S;
Y is H, OH or NH₂;
Z₁, Z₂ and Z₃ each independently is O⁻ or BH₃⁻;
W₁ and W₂ each independently is O, CH₂, C(Hal)₂ or NH, wherein Hal is halogen, preferably F or Cl;
n is 0 or 1, provided that when n is 0 and W₂ is O, Z₁ is BH₃⁻; and when n is 1, at least one of W₁ and W₂ is not O;
m is 3 or 4; and
B⁺ represents a pharmaceutically acceptable cation, and diastereoisomers thereof,
but excluding the compounds wherein n is 0, Z₁ and Z₃ are each O⁻, and W₂ is CH₂ or NH, and the compounds wherein n is 1 and Z₁ to Z₃ are each O⁻.

2. The compound of claim 1, wherein n is 0, and Z₁ and Z₃ are 0; or n is 0, and at least one of Z₁ and Z₃ is BH₃⁻; or n is 1, and at least one of Z₁ to Z₃ is BH₃⁻.

3. The compound of claim 2, wherein n is 0, comprising a sole borano group at position α, namely, Z₁ is BH₃⁻, and Z₂ is O⁻; or at position β, namely, Z₃ is BH₃⁻ and Z₁ is O⁻, or two borano groups at positions α,β, namely, Z₁ and Z₃ are BH₃⁻.

4. The compound of claim 2, wherein n is 1, comprising a sole borano group at position α, namely, Z₁ is BH₃⁻, and Z₂ and Z₃ are O⁻; at position β, namely, Z₂ is BH₃⁻, and Z₁ and Z₃ are O⁻, or at position γ, namely, Z₃ is BH₃⁻, and Z₁ and Z₂ are O⁻, two borano groups at positions α and β, namely, Z₁ and Z₂ are BH₃⁻, and Z₃ is O⁻; at positions α and γ, namely, Z₁ and Z₃ are BH₃⁻, and Z₂ is O⁻; or at positions β and γ, namely, Z₂ and Z₃ are BH₃⁻, and Z₁ is O⁻, or three borano groups at positions α,β and γ, namely, Z₁ to Z₃ are BH₃⁻.

5. The compound of claim 1, wherein X is an adenine residue, wherein R₁ is H, halogen, O-hydrocarbyl or S-hydrocarbyl; R₂ and R₃ each independently is H or hydrocarbyl; Y is OH; n is 1; Z₁ is BH₃⁻; Z₂ and Z₃ are O⁻; W₁ is O; and W₂ is CH₂, CF₂ or CCl₂.

6. The compound of claim 1, wherein X is an adenine residue, wherein R₁ is H or NR₄R₅, and R₄ and R₅ each independently is H or hydrocarbyl or R₄ and R₅ together with the nitrogen atom to which they are attached form a saturated or unsaturated heterocyclic ring optionally containing 1-2 further heteroatoms selected from oxygen, nitrogen or sulfur; R₂ and R₃ each independently is H or hydrocarbyl; Y is OH; n is 1; Z₁ is BH₃⁻; Z₂ and Z₃ are O⁻; W₁ is O; and W₂ is CH₂, CF₂ or CCl₂.

7. The compound of claim 1, wherein X is an adenine residue, wherein R₁ is H, halogen, O-hydrocarbyl or S-hydrocarbyl; R₂ and R₃ each independently is H or hydrocarbyl; Y is OH; n is 0; and (i) Z₁ and Z₃ are O⁻; and W₂ is CF₂ or CCl₂; or (ii) Z₁ is BH₃⁻; and W₂ is O.

8. The compound of claim 1, wherein X is an adenine residue, wherein R₁ is H or NR₄R₅, and R₄ and R₅ each independently is H or hydrocarbyl or R₄ and R₅ together with the nitrogen atom to which they are attached form a saturated or unsaturated heterocyclic ring optionally containing 1-2 further heteroatoms selected from oxygen, nitrogen or sulfur; R₂ and R₃ each independently is H or hydrocarbyl; Y is OH; n is 0; Z₁ and Z₃ are O⁻; and W₂ is CF₂ or CCl₂.

9. The compound of claim 5, wherein X is an adenine residue wherein R₁ is H, R₂ and R₃ are H, Y is OH, n is 1, Z₁ is BH₃⁻, Z₂ and Z₃ are O⁻, W₁ is O, and W₂ is CH₂ (compound 3).

10. The compound of claim 5, wherein X is an adenine residue wherein R₁ is SMe, R₂ and R₃ are H, Y is OH, n is 1, Z₁ is BH₃⁻, Z₂ and Z₃ are O⁻, W₁ is O, and W₂ is CH₂ (compound 4).

11. The compound of claim 10, characterized by being the isomer with a retention time (Rt) of 5.57 min when separated from a mixture of diastereoisomers using a semi-preparative reverse-phase Gemini 5u column (C-18 110A, 250×10 mm, 5 micron), and isocratic elution [100 mM triethylammonium acetate, pH 7: MeOH, 85:15] with flow rate of 5 ml/min (compound 4B).

12. The compound of claim 5, wherein X is an adenine residue wherein R₁ is SMe, R₂ and R₃ are H, Y is OH, n is 1, Z₁ is BH₃⁻, Z₂ and Z₃ are O⁻, W₁ is O; and W₂ is CCl₂ or CF₂ (compounds 21 and 22, respectively).

13. The compound of claim 7, wherein X is an adenine residue wherein R₁ is SMe, R₂ and R₃ are H, Y is OH, n is 0, Z₁ and Z₃ are O⁻, and W₂ is CCl₂ or CF₂ (compounds 17 and 18, respectively).

14. The compound of claim 7, wherein X is an adenine residue wherein R₁ is SMe, R₂ and R₃ are H, Y is OH, n is 0, Z₁ is BH³⁻, Z₃ is O⁻, and W₂ is O (compound 19).

15. The compound of claim 14, characterized by being the isomer with a retention time (Rt) of 8.073 min when separated from a mixture of diastereoisomers using a semi-preparative reverse-phase Gemini 5u column (C-18 110A, 250×10 mm, 5 micron), and isocratic elution [100 mM triethylammonium acetate, pH 7: acetonitrile, 88:12] with flow rate of 1 ml/min (compound 19A).

16. The compound of claim 1, wherein X is an uracil residue, wherein R₆ is H, halogen, O-hydrocarbyl or S-hydrocarbyl; R₇ is O or S; Y is OH; n is 1; Z₁ is BH₃⁻; Z₂ and Z₃ are O⁻; W₁ is O; and W₂ is CH₂, CF₂ or CCl₂.

17. The compound of claim 1, wherein X is an uracil residue, wherein R₆ is H or NR₈R₉, and R₈ and R₉ each independently is H or hydrocarbyl or R₈ and R₉ together with the nitrogen atom to which they are attached form a saturated or unsaturated heterocyclic ring optionally containing 1-2 further heteroatoms selected from oxygen, nitrogen or sulfur; R₇ is O or S; Y is OH; n is 1; Z₁ is BH₃⁻; Z₂ and Z₃ are O⁻; W₁ is O; and W₂ is CH₂, CF₂ or CCl₂.

18. The compound of claim 1, wherein X is an uracil residue, wherein R₆ is H, halogen, O-hydrocarbyl or S-hydrocarbyl; R₇ is O or S; Y is OH; n is 0; Z₁ and Z₃ are O⁻; and W₂ is CF₂ or CCl₂.

19. The compound of claim 1, wherein X is an uracil residue, wherein R₆ is H or NR₈R₉, and R₈ and R₉ each independently is H or hydrocarbyl or R₈ and R₉ together with the nitrogen atom to which they are attached form a saturated or unsaturated heterocyclic ring optionally containing 1-2 further heteroatoms selected from oxygen, nitrogen or sulfur; R₇ is O or S; Y is OH; n is 0; Z₁ and Z₃ are O⁻; and W₂ is CF₂ or CCl₂.

20. The compound of claim 1, wherein B is a cation of an alkali metal, NH₄⁺, an organic cation of the formula R₄N⁺ wherein each one of the Rs independently is H or C₁-C₂₂, preferably C₁-C₆, alkyl, a cationic lipid or a mixture of cationic lipids.

21. A pharmaceutical composition comprising a compound of the general formula I as claimed in claim 1 or a pharmaceutically acceptable salt thereof, and a pharmaceutically acceptable carrier or diluent.

22. A pharmaceutical composition for treatment of a disease, disorder or condition modulated by P2Y receptors, comprising a compound of the general formula I in claim 1 or a pharmaceutically acceptable salt thereof, and a pharmaceutically acceptable carrier or diluent.

23. The pharmaceutical composition of claim 22, wherein said disease or disorder modulated by P2Y receptors is type 2 diabetes.

24. The pharmaceutical composition of claim 22, for pain control.

25. The pharmaceutical composition of any one of claims 22 to 24, comprising a compound selected from compounds 4B, 17, 18, 19A, 21A or 21B, or the compound of the general formula I, wherein X is an adenine residue wherein R₁ is SMe, R₂ and R₃ are H, Y is OH, n is 1, Z₁ to Z₃ are O⁻, W₁ is O, and W₂ is CH₂ (compound 2).

26. Use of a compound of the general formula I in claim 1 or a pharmaceutically acceptable salt thereof for the preparation of a pharmaceutical composition for treatment of a disease, disorder or condition modulated by P2Y receptors.

27. A compound of the general formula I in claim 1 or a pharmaceutically acceptable salt thereof for treatment of a disease, disorder or condition modulated by P2Y receptors.

28. A method for treatment of a disease, disorder or condition modulated by P2Y receptors in an individual in need, comprising administering to said individual an effective amount of a compound of the general formula I in claim 1 or a pharmaceutically acceptable salt thereof.

29. The method of claim 28, wherein said disease or disorder modulated by P2Y receptors is type 2 diabetes.

30. The method of claim 28, for controlling pain in an individual in need.
